SEQUENCE LISTING (22 sequences)

SEQ ID NO: 1 - protein, 744 aa, synthetic construct
  REGION 1..744: note = Exemplary S309-CAR
MEFGLSWLFLVAILKGVQCVDQVQLVQSGAEVKKPGASVKVSCKASGYPFTSYGISWVRQAPGQGLEWMGWISTYNGNTNYAQKFQGRVTMTTDTSTTTGYMELRRLRSDDTAVYYCARDYTRGAWFGESLIGGFDNWGQGTLVTVSSGGGGSGGGGSGGGGSGGGGSEIVLTQSPGTLSLSPGERATLSCRASQTVSSTSLAWYQQKPGQAPRLLIYGASSRATGIPDRFSGSGSGTDFTLTISRLEPEDFAVYYCQQHDTSLTFGGGTKVEIKSYVTVSSQDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 2 - protein, 20 aa, synthetic construct
  REGION 1..20: note = E2A protein
QCTNYALLKLAGDVESNPGP

SEQ ID NO: 3 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 4 - protein, 926 aa, synthetic construct
  REGION 1..926: note = Exemplary S309-CAR-IL-15
MEFGLSWLFLVAILKGVQCVDQVQLVQSGAEVKKPGASVKVSCKASGYPFTSYGISWVRQAPGQGLEWMGWISTYNGNTNYAQKFQGRVTMTTDTSTTTGYMELRRLRSDDTAVYYCARDYTRGAWFGESLIGGFDNWGQGTLVTVSSGGGGSGGGGSGGGGSGGGGSEIVLTQSPGTLSLSPGERATLSCRASQTVSSTSLAWYQQKPGQAPRLLIYGASSRATGIPDRFSGSGSGTDFTLTISRLEPEDFAVYYCQQHDTSLTFGGGTKVEIKSYVTVSSQDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRQCTNYALLKLAGDVESNPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 5 - DNA, 2232 bp, synthetic construct
  misc_feature 1..2232: note = Exemplary S309-CAR nucleic acid
  source 1..2232: mol_type = other DNA
atggagtttgggctgagctggctttttcttgtggctattttaaaaggtgtccagtgcgtcgaccaggtgcagctggtgcagagcggcgcggaagtgaaaaaaccgggcgcgagcgtgaaagtgagctgcaaagcgagcggctatccgtttaccagctatggcattagctgggtgcgccaggcgccgggccagggcctggaatggatgggctggattagcacctataacggcaacaccaactatgcgcagaaatttcagggccgcgtgaccatgaccaccgataccagcaccaccaccggctatatggaactgcgccgcctgcgcagcgatgataccgcggtgtattattgcgcgcgcgattatacccgcggcgcgtggtttggcgaaagcctgattggcggctttgataactggggccagggcaccctggtgaccgtgagcagcggtggtggtggttctggtggtggtggttctggcggcggcggctccggtggtggtggatccgaaattgtgctgacccagagcccgggcaccctgagcctgagcccgggcgaacgcgcgaccctgagctgccgcgcgagccagaccgtgagcagcaccagcctggcgtggtatcagcagaaaccgggccaggcgccgcgcctgctgatttatggcgcgagcagccgcgcgaccggcattccggatcgctttagcggcagcggcagcggcaccgattttaccctgaccattagccgcctggaaccggaagattttgcggtgtattattgccagcagcatgataccagcctgacctttggcggcggcaccaaagtggaaattaaatcgtacgtcaccgtctcttcacaggatcccgccgagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaaaagatcccaaattttgggtgctggtggtggttggtggagtcctggcttgctatagcttgctagtaacagtggcctttattattttttgggtgaggagtaagaggagcaggctcctgcacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 6 - DNA, 60 bp, synthetic construct
  misc_feature 1..60: note = E2A nucleic acid
  source 1..60: mol_type = other DNA
cagtgtactaattatgctctcttgaaattggctggagatgttgagagcaatcccgggccc

SEQ ID NO: 7 - DNA, 489 bp, Homo sapiens
  source 1..489: mol_type = unassigned DNA
atgcggatcagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagctga

SEQ ID NO: 8 - DNA, 2781 bp, synthetic construct
  misc_feature 1..2781: note = Exemplary S309-CAR-IL-15 nucleic acid
  source 1..2781: mol_type = other DNA
atggagtttgggctgagctggctttttcttgtggctattttaaaaggtgtccagtgcgtcgaccaggtgcagctggtgcagagcggcgcggaagtgaaaaaaccgggcgcgagcgtgaaagtgagctgcaaagcgagcggctatccgtttaccagctatggcattagctgggtgcgccaggcgccgggccagggcctggaatggatgggctggattagcacctataacggcaacaccaactatgcgcagaaatttcagggccgcgtgaccatgaccaccgataccagcaccaccaccggctatatggaactgcgccgcctgcgcagcgatgataccgcggtgtattattgcgcgcgcgattatacccgcggcgcgtggtttggcgaaagcctgattggcggctttgataactggggccagggcaccctggtgaccgtgagcagcggtggtggtggttctggtggtggtggttctggcggcggcggctccggtggtggtggatccgaaattgtgctgacccagagcccgggcaccctgagcctgagcccgggcgaacgcgcgaccctgagctgccgcgcgagccagaccgtgagcagcaccagcctggcgtggtatcagcagaaaccgggccaggcgccgcgcctgctgatttatggcgcgagcagccgcgcgaccggcattccggatcgctttagcggcagcggcagcggcaccgattttaccctgaccattagccgcctggaaccggaagattttgcggtgtattattgccagcagcatgataccagcctgacctttggcggcggcaccaaagtggaaattaaatcgtacgtcaccgtctcttcacaggatcccgccgagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaaaagatcccaaattttgggtgctggtggtggttggtggagtcctggcttgctatagcttgctagtaacagtggcctttattattttttgggtgaggagtaagaggagcaggctcctgcacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccagtgtactaattatgctctcttgaaattggctggagatgttgagagcaatcccgggcccatgcggatcagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagctga

SEQ ID NO: 9 - DNA, 60 bp, synthetic construct
  misc_feature 1..60: note = SARS-CoV-2 S forward primer
  source 1..60: mol_type = other DNA
tctagagattacaaggatgacgacgataagtaactcgagatcgatccggattagtccaat

SEQ ID NO: 10 - DNA, 29 bp, synthetic construct
  misc_feature 1..29: note = SARS-CoV-2 S reverse primer
  source 1..29: mol_type = other DNA
gtcgacgcactggacaccttttaaaatag

SEQ ID NO: 11 - DNA, 51 bp, synthetic construct
  misc_feature 1..51: note = pSFG_SARS-CoV2-Sdelta _Forward primer
  source 1..51: mol_type = other DNA
tcatgcggcagctgttgctctagagattacaaggatgacgacgataagtaa

SEQ ID NO: 12 - DNA, 48 bp, synthetic construct
  misc_feature 1..48: note = pSFG_SARS-CoV2-Sdelta_Reverse primer
  source 1..48: mol_type = other DNA
caccaagaacacaaacatgtcgacgcactggacaccttttaaaatagc

SEQ ID NO: 13 - DNA, 48 bp, synthetic construct
  misc_feature 1..48: note = pLV_ SARS-CoV-2 Sdelta_Forward primer
  source 1..48: mol_type = other DNA
ggtgtccagtgcgtcgacatgtttgtgttcttggtgttgcttccactg

SEQ ID NO: 14 - DNA, 40 bp, synthetic construct
  misc_feature 1..40: note = pLV_SARS-CoV-2 Sdelta_Reverse primer
  source 1..40: mol_type = other DNA
atccttgtaatctctagagcaacagctgccgcatgagcag

SEQ ID NO: 15 - DNA, 51 bp, synthetic construct
  misc_feature 1..51: note = pSFG_ SARS-CoV2-S delta+_Forward primer
  source 1..51: mol_type = other DNA
tcctgcggcagctgctgctctagagattacaaggatgacgacgataagtaa

SEQ ID NO: 16 - DNA, 48 bp, synthetic construct
  misc_feature 1..48: note = pSFG_SARS-CoV-2 Sdelta+_Reverse primer
  source 1..48: mol_type = other DNA
gaccaggaagacaaacatgtcgacgcactggacaccttttaaaatagc

SEQ ID NO: 17 - DNA, 43 bp, synthetic construct
  misc_feature 1..43: note = pCAG_ SARS-CoV2-S delta+_Forward primer
  source 1..43: mol_type = other DNA
ggtgtccagtgcgtcgacatgtttgtcttcctggtcctgctgc

SEQ ID NO: 18 - DNA, 40 bp, synthetic construct
  misc_feature 1..40: note = pCAG_SARS-CoV-2 Sdelta+_Reverse
  source 1..40: mol_type = other DNA
atccttgtaatctctagagcagcagctgccgcaggagcag

SEQ ID NO: 19 - DNA, 51 bp, synthetic construct; identical to SEQ ID NO: 15
  misc_feature 1..51: note = pSFG_SARS-CoV-2 Smu_Forward primer
  source 1..51: mol_type = other DNA

SEQ ID NO: 20 - DNA, 48 bp, synthetic construct; identical to SEQ ID NO: 16
  misc_feature 1..48: note = pSFG_SARS-CoV-2 Smu_Reverse primer
  source 1..48: mol_type = other DNA

SEQ ID NO: 21 - DNA, 43 bp, synthetic construct; identical to SEQ ID NO: 17
  misc_feature 1..43: note = pCAG_ SARS-CoV-2 Smu_Forward primer
  source 1..43: mol_type = other DNA

SEQ ID NO: 22 - DNA, 40 bp, synthetic construct; identical to SEQ ID NO: 18
  misc_feature 1..40: note = pCAG_ SARS-CoV-2 Smu_Reverse primer
  source 1..40: mol_type = other DNA

(19) United States
(12) Patent Application Publication
(10) Pub. No.: US 2024/0415966 A1
(43) Pub. Date: Dec. 19, 2024

(54) S309 CHIMERIC ANTIGEN RECEPTORS AND METHODS OF USE

(71) Applicant: Rutgers, The State University of New Jersey, New Brunswick, NJ (US)

(72) Inventors: Dongfang Liu, Millburn, NJ (US); Minh Ma, New Brunswick, NJ (US); Saiaditya Badeti, New Brunswick, NJ (US)

(73) Assignee: Rutgers, The State University of New Jersey, New Brunswick, NJ (US)

(21) Appl. No.: 18/821,512

(22) Filed: Aug. 30, 2024

Related U.S. Application Data
(62) Division of application No. 17/551,006, filed on Dec. 14, 2021, now Pat. No. 12,109,237.
(60) Provisional application No. 63/125,820, filed on Dec. 15, 2020.

(51) Int. Cl. A61P 31/14; A61K 38/00; A61K 39/00; C07K 14/54; C07K 14/705; C07K 14/715; C07K 14/725; C07K 16/10

(52) U.S. Cl. CPC A61P 31/14; A61K 39/4613; A61K 39/4631; A61K 39/464838; A61K 2239/28; C07K 14/5443; C07K 14/7051; C07K 14/70521; C07K 14/7151; C07K 16/1003; A61K 38/00; C07K 2317/565; C07K 2317/622

(57) ABSTRACT

Chimeric antigen receptors (CARs) including an scFv binding to coronavirus spike protein (S309 scFv), nucleic acids encoding the CARs, vectors including nucleic acids encoding the CARs, and cells expressing the CARs are provided. Methods of treating a subject with coronavirus are also provided, including administering to the subject a modified immune cell expressing a disclosed CAR.

### CROSS REFERENCE TO RELATED APPLICATIONS

This application is a divisional of U.S. application Ser. No. 17/551,006, filed Dec. 14, 2021, which in turn claims the benefit of U.S. Provisional Application No. 63/125,820, filed Dec. 15, 2020, both of which are incorporated herein by reference in their entirety.

### ACKNOWLEDGMENT OF GOVERNMENT SUPPORT

This invention was made with government support under grant number AI130197 awarded by the National Institutes of Health. The government has certain rights in the invention.

### FIELD

This disclosure relates to chimeric antigen receptors, particular chimeric antigen receptors targeting coronavirus spike protein, and methods of their use for treating coronavirus infection.

### SEQUENCE LISTING INCORPORATION

The Sequence Listing is submitted as an XML file in the form of the file named 7213-105542-03_Sequence_Listing, which was created on Aug. 30, 2024, and is 35,908 bytes, which is incorporated by reference herein.

### BACKGROUND

Severe Acute Respiratory Syndrome Coronavirus 2 (SARS-COV-2) is highly contagious, and is now widespread throughout the world. The disease caused by SARS-COV-2 (COVID-19) presents severe symptoms including pneumonia, acute respiratory distress syndrome, neurological symptoms, organ failure, and death. More importantly, severe COVID-19 patients may experience dysregulation of an appropriate immune response, characterized by lymphopenia, high neutrophil levels, and increased pro-inflammatory cytokines and chemokines (Song et al., Nat. Commun. 11:3410, 2020).

Current treatments for COVID-19 patients can be classified into three categories: anti-viral treatments, immunosuppression-based treatments, and other supporting treatments such as convalescent plasma. Specifically, in a few trials patients have been given combinations of antivirals including umifenovir (Xu et a., Mil. Med. Res. 7:22, 2020), remdesivir/ribavirin (Jean et al., J. Microbiol. Immunol. Infect. 53:436-443, 2020), chloroquine (Shah et al., Int. J. Rheum. Dis. 23:613-619, 2020), the chloroquine analog hydroxychloroquine (Chowdhury et al., Acad. Emerg. Med. 27:493-504, 2020), and/or lopinavir/ritonavir (Dong et al., Drug Discov. Ther. 15:58-60, 2020; Russell et al., Ecancermedicalscience 14:1022, 2020). Non-steroidal anti-inflammatory drugs (NSAIDs), antibodies against IL-6 receptors, and corticosteroids have also been used during the early acute phase of SARS-COV-2 to suppress the overactivated immune response (Dong et al., Drug Discov. Ther. 15:58-60, 2020). Other supporting therapies including supplemental oxygen and mechanical ventilatory support have also been used when indicated (e.g., intubation, etc.).

Natural killer (NK) cells have been shown to participate in the first line of defense in humans and mice against pathogen-infected or malignant cells. There are three known defense mechanisms by which NK cells use to mediate killing: production of cytokines such as IFN-γ to stimulate other direct antiviral mechanisms, release of lytic granules, (stored effector molecules such as perforin and granzymes) by direct binding through stimulatory receptors, and induction of apoptosis through the interaction of TNF-related apoptosis induction ligand (TRAIL) on NK cells to the death receptors on the target cells.

Clinically, natural killer (NK) cells were first defined as CD56^{bright}CD 16⁻ and CD56^{dim}CD16^{+cells} in the peripheral blood. NK cells isolated from peripheral blood can be further modified to express chimeric antigen receptors (CARs) for treating a variety of cancers and infectious diseases (Liu et al., Protein Cell 8:861-877, 2017). Recent preclinical studies of CAR-NK cells in cancer immunotherapy show several advantages over CAR-T cells in clinical safety. Unlike CAR-T, CAR-NK cells do not present additional risk for the development of severe graft-versus-host-disease (GVHD). More importantly, CAR-NK cells are associated with reduced host cytotoxicity compared to CAR-T cells. In particular, NK cells are less likely to induce cytokine release syndrome (CRS) that could potentially exacerbate COVID-19 symptoms in severe patients (Shah et al., Br. J. Haematol. 177:457-466, 2017).

### SUMMARY

Previous studies show that the genome sequence of SARS-COV-2 is 77% identical to that of SARS-COV (Zhou et al., Nature 579:270-273, 2020). Several neutralizing antibodies were isolated from memory B cells of convalescent SARS patients. One of these, named S309, potently neutralizes both pseudotyped SARS-COV-2 viral particles and authentic SARS-COV-2 by binding to both the ‘closed’ and ‘open’ ectodomain trimer conformations of the SARS-COV-2 Spike glycoprotein (Pinto et al., Nature 583:290-295, 2020).

Provided herein is a novel approach for the generation of CAR-NK cells for targeting SARS-COV-2 using the scFv domain of S309. Compared to previously generated Spike protein-targeting CAR-NK cells, S309-CAR-NK cells show superior killing activities against pseudotyped SARS-COV-2 virus. This demonstrates that ‘off-the-shelf’ S309-CAR-NK cells may have the potential to prevent SARS-COV-2 infection, as well as to treat immunocompromised patients or those with comorbidities such as diabetes, malnutrition, and certain genetic disorders.

In some embodiments, disclosed is a chimeric antigen receptor including an antigen binding domain that specifically binds coronavirus spike protein, such as an scFv of antibody S309; a hinge domain; a transmembrane domain; and an intracellular domain. In some embodiments, the antigen binding domain includes the variable heavy chain (VH) domain complementarity determining region 1 (CDR1), CDR2 and CDR3 amino acid sequences of amino acid positions 47-54, 72-79, and 118-137 of SEQ ID NO: 1, respectively, and the variable light chain (VL) domain CDR1, CDR2 and CDR3 amino acid sequences of amino acid positions 195-201, 219-221, and 258-265 of SEQ ID NO: 1, respectively. In some examples, the antigen binding domain has at least 90% sequence identity amino acids 22-275 of SEQ ID NO: 1, or includes or consists of amino acids 22-275 of SEQ ID NO: 1. In some examples, the hinge domain comprises an IgG1 domain, the transmembrane domain comprises a CD28 transmembrane domain, and the intracellular domain comprises a CD28 domain, a 4-1BB domain, and a CD3ζ domain. In additional examples, the chimeric antigen receptor further includes an interleukin-15 (IL-15) domain. In non-limiting examples, the chimeric antigen receptor has at least 90% sequence identity to the amino acid sequence SEQ ID NO: 1 or SEQ ID NO: 4, or includes or consists of the amino acid sequence of SEQ ID NO: 1 or SEQ ID NO: 4.

Also provided are nucleic acids encoding the chimeric antigen receptors disclosed herein. In some embodiments, the antigen binding domain is encoded by a nucleic acid including the variable heavy chain (VH) domain complementarity determining region 1 (CDR1), CDR2 and CDR3 nucleic acid sequences of nucleic acid positions 139-162, 514-237, and 352-411 of SEQ ID NO: 5, respectively, and the variable light chain (VL) domain CDR1, CDR2 and CDR3 nucleic acid sequences of nucleic acid positions 583-603, 655-663, and 772-765 of SEQ ID NO: 5, respectively. In some embodiments, the nucleic acid encoding the S309 scFv is codon-optimized. In particular examples, the nucleic acid encoding the CAR has at least 90% sequence identity to the nucleic acid sequence of SEQ ID NO: 5 or SEQ ID NO: 8, or comprises or consists of the amino acid sequence of SEQ ID NO: 5 or SEQ ID NO: 8. The nucleic acids are included in a vector (such as a retroviral vector) in some embodiments.

Modified immune cells, for example, natural killer (NK) cells or T cells expressing the disclosed chimeric antigen receptor are provided. In some embodiments, the modified NK cell is an NK-92 cell or NK-92MI cell.

Also provided are methods of treating a subject having or suspected of having a coronavirus infection, including administering an effective amount of a modified NK cell expressing a disclosed CAR to the subject. In particular examples, the subject is infected with SARS-COV-1 or SARS-COV-2.

The foregoing and other features of the disclosure will become more apparent from the following detailed description, which proceeds with reference to the accompanying figures.

### BRIEF DESCRIPTION OF THE DRAWINGS

FIGS. 1A-1C show generation of S309-CAR-NK-92MI cells. FIG. 1A is a schematic diagram of an exemplary plasmid construct of a S309-CAR. The SFG retroviral vector contains the S309 single chain antibody fragment (PDB accession code 6WS6), a human IgG1 CH2CH3 hinge region, a CD28 transmembrane region, followed by the co-stimulatory CD28 and 4-1BB domains, and the intracellular domain of CD3ζ. FIG. 1B shows determination of S309-CAR-NK expression by flow cytometry. S309-CAR cells were collected and stained with anti-CD56 and CAR F(ab)2 domain [IgG (H+L)] for flow cytometry. The cells were then sorted to achieve a homogenous population of high CAR expression. FIG. 1C illustrates immunoprofiling of S309-CAR-NK by flow cytometry. S309-CAR and the wildtype NK-92MI cells were stained with antibodies against different immunomodulatory receptors including CTLA4, PD1, NKG2A, NKG2C, NKG2D, NKp46, CD56, CD16, 2B4, DNAM-1, CD94, KLRG1, TIM3, LAG3, and TIGIT.

FIGS. 2A-2D illustrate that S309-CAR-NK-92MI cells bind to RBD domain of SARS-COV-2 S protein and pseudotyped SARS-COV-2 viral particles. FIG. 2A is representative dot plots showing the efficiency of S309-CAR binding to SARS-COV-2-RBD. S309-CAR or NK-92MI cells were incubated with the RBD recombinant protein of SARS-COV-1 (left) or SARS-COV-2 (right). FIG. 2B is a schematic diagram illustrating generation of pseudotyped SARS-COV-2 viral particles. 293T cells were transfected with the indicated plasmids for 72 hours for the generation of pseudotyped SARS-COV-2 viral particles. FIG. 2C shows representative histograms showing S309-CAR-NK binds to the pseudotyped SARS-COV-2 viral particles. S309-CAR-NK or NK-92MI or 293T-hACE2 cells were incubated with pseudotyped SARS-COV-2 viral particles, S1 subunit, or full-length S recombinant protein at 37° C. for 1 hour. Cells were then harvested and stained with anti-S1 subunit and evaluated by flow cytometry. The experimental sample was performed in triplicates with MFI=13579±251 (a.u.). FIG. 2D shows quantitative data of the binding efficiency of S309-CAR-NK to pseudotyped SARS-COV-2 viral particles. The experimental sample was performed in triplicates with binding efficiency of over 90%. Data represent mean±standard error of the mean (SEM) of three independent experiments. Unpaired Student's t test was employed. ****p<0.0001.

FIGS. 3A-3D demonstrate increased CD107a surface expression and killing activity of S309-CAR-NK-92MI cells against 293T-hACE2-RBD and A549-Spike. FIG. 3A is a schematic diagram showing generation of transient 293T-hACE2-RBD and stable A549-Spike cell lines. 293T-hACE2 cells were transfected with RBD-containing plasmid for 48 hours. Transfected 293T-hACE2-RBD cells were then harvested. For the generation of A549-Spike, 293T cells were transfected with the retrovirus transfection system for 48 hours. The spike retrovirus was filtered and transduced into A549 cells for an additional 48-72 hours. FIG. 3B shows representative dot plots showing the expression of RBD or Spike in 293T-hACE2 (top) or A549 cell (bottom). 293T-hACE2-RBD and A549-Spike cells were stained with anti-RBD and the expression was confirmed by flow cytometry. The stable A549-Spike cell line was then sorted to achieve high levels of spike expression. FIG. 3C shows quantitative data of CD107a surface expression assay of S309-CAR-NK against 293T-hACE2-RBD or A549-Spike cell lines. Briefly, S309-CAR-NK-92MI cells were cocultured with either 293T-hACE2-RBD cells, A549-Spike cells, stimulated with PMA/Ionomycin, or incubated alone for 2 hours at 37° C. Cells were then harvested and stained for CAR F(ab)2 domain [IgG (H+L)] and CD107a. Data represent mean±SEM from two experiments. FIG. 3D shows a 4-hour gold standard Cr⁵¹ release assay of S309-CAR-NK and NK-92MI against various target cell lines. 293T-hACE2-RBD (top), A549-Spike (middle), and HepG2 (bottom) cell lines were used as target cells for S309-CAR-NK and NK-92MI. Experimental groups were performed in triplicates. Error bars represent mean±SEM from at least two independent experiments. Unpaired Student's t test was used for both panels FIG. 3C and FIG. 3D. ns p>0.05, *p<0.05, **p<0.01, ***p<0.001, and ****p<0.0001.

FIGS. 4A-4D illustrate increased killing activity of expanded primary S309-CAR-NK against A549-Spike cell line. FIG. 4A is a schematic representation of human primary S309-CAR-NK expansion system. Briefly, irradiated (100 Gy) 221-mIL21 feeder cells were cocultured with PBMC supplemented with IL-2 and IL-15 on Day 0. In parallel, 293T cells were transfected with the retrovirus packaging system to produce S309-CAR retrovirus that were then transduced into the expanded PBNK cells in the presence of IL-2 and IL-15. Primary S309-CAR-NK cells were harvested on Day 7 and continued expansion for 21 days. FIG. 4B shows representative dot plots of expanded primary NK cells and primary S309-CAR-NK. The purity of NK cells and the expression of CAR were monitored every 3-4 days. FIG. 4C shows immunophenotyping of primary S309-CAR-NK cells using flow cytometry. Antibodies against various immunomodulatory receptors including CTLA4, PD1, NKG2A, CD56, CD16, 2B4, NKG2C, NKG2D, NKp46, DNAM-1, CD94, TIGIT, KLRG1, TIM3, and LAG3 were used to stain both primary NK cells and S309-CAR-NK. FIG. 4D is a graph showing quantitative data of cytotoxic activity of primary S309-CAR-NK against A549-Spike. Briefly, expanded S309-CAR-NK cells were blocked with anti-CD16 for 30 minutes and then anti-NKG2D for 30 minutes on ice. The target cells were labeled with Cr⁵¹ for 2 hours prior to coculturing with primary S309-CAR-NK cells for an additional 4 hours. The experiment was repeated at least twice. Error bars represent SEM. Unpaired Student's t test was used. **p<0.01, ***p<0.001, and ****p<0.0001.

FIGS. 5A-5D show a comparison of S309-CAR-NK cells and CR3022-CAR-NK cells. FIG. 5A is a diagram of S309 and CR3022 neutralizing antibodies binding to different epitopes of the SARS-COV-2 S protein. Both open and closed conformation states of SARS-COV-2 S protein are shown. FIG. 5B shows quantitative data of CD107a surface expression of both S309-CAR-NK-92MI and CR3022-CAR-NK-92MI. Both transient 293T-hACE2-RBD and stable A549-Spike cell lines were used as target cells. Error bars represent SEM from at least two independent experiments. FIG. 5C shows a comparison of killing activity of S309-CAR and CR3022-CAR using the 4-hour Cr⁵¹ release assay. Effector cells were cocultured with Cr⁵¹-labeled target cells at 37° C. for 4 hours. The assay was repeated for at least two times per target cell line. FIG. 5D shows that expanded primary S309-CAR-NK cells have increased killing activity against A549-Spike cells compared to primary CR3022-CAR-NK. Effector cells were blocked with anti-CD16 and anti-NKG2D prior to coculturing with A549-Spike target cells for 4 hours at 37° C. Data were pooled from three independent experiments. Unpaired Student's t test was employed for all panels. ns p>0.05, *p<0.05, **p<0.01, ***p<0.001, and ****p<0.0001.

FIGS. 6A and 6B illustrate that primary S309-CAR-NK cells effectively bind to all existing variants of SARS-COV-2 pseudotyped virus. FIG. 6A is a representative histogram showing the SARS-COV-2 pseudovirus binding efficiency of primary S309-CAR-NK cells. FIG. 6B is a bar graph showing the SARS-COV-2 pseudovirus binding efficiency of primary S309-CAR-NK cells. Primary un-transduced NK cells or S309-CAR-NK cells, or 293T-hACE2 cells were incubated with SARS-COV-2 pseudotyped virus for 2 hours at 37° C. prior to staining with anti-spike and flow cytometry. Error bars represent±SEM. Experiment was repeated three times.

FIG. 7 is a representative histogram of A549 cell lines expressing Sα, Sδ, Sδ+, or Sμ protein pre-sorting. A549 cells were transduced using plasmids containing Sα, Sδ, Sδ+, or Sμ in SFG vector. Transduced cells were harvested, and the expression was confirmed by flow cytometry.

### SEQUENCE LISTING

Any nucleic acid and amino acid sequences listed herein or in the accompanying Sequence Listing are shown using standard letter abbreviations for nucleotide bases and amino acids, as defined in 37 C.F.R. § 1.822. In at least some cases, only one strand of each nucleic acid sequence is shown, but the complementary strand is understood as included by any reference to the displayed strand.

SEQ ID NO: 1 is the amino acid sequence of an exemplary S309-CAR:

MEFGLSWLFLVAILKGVQCVDQVQLVQSGAEVKKPGASVKVSCKASGYPF
TSYGISWVRQAPGQGLEWMGWISTYNGNTNYAQKFQGRVTMTTDTSTTTG
YMELRRLRSDDTAVYYCARDYTRGAWFGESLIGGFDNWGQGTLVTVSSGG
GGSGGGGSGGGGSGGGGSEIVLTQSPGTLSLSPGERATLSCRASQTVSST
SLAWYQQKPGQAPRLLIYGASSRATGIPDRFSGSGSGTDFTLTISRLEPE
DFAVYYCQQHDTSLTFGGGTKVEIKSYVTVSSQDPAEPKSPDKTHTCPPC
PAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALP
APIEKTISKAKGQPREPQVYTLPPSRDELTKNQVSLTCLVKGFYPSDIAV
EWESNGQPENNYKTTPPVLDSDGSFFLYSKLTVDKSRWQQGNVFSCSVMH
EALHNHYTQKSLSLSPGKKDPKFWVLVVVGGVLACYSLLVTVAFIIFWVR
SKRSRLLHSDYMNMTPRRPGPTRKHYQPYAPPRDFAAYRSKRGRKKLLYI
FKQPFMRPVQTTQEEDGCSCRFPEEEEGGCELRVKFSRSADAPAYQQGQN
QLYNELNLGRREEYDVLDKRRGRDPEMGGKPRRKNPQEGLYNELQKDKMA
EAYSEIGMKGERRRGKGHDGLYQGLSTATKDTYDALHMQALPPR

SEQ ID NO: 2 is the amino acid sequence of an exemplary E2A protein:

QCTNYALLKLAGDVESNPGP

SEQ ID NO: 3 is the amino acid sequence of an exemplary interleukin-15 protein:

MRISKPHLRSISIQCYLCLLLNSHFLTEAGIHVFILGCFSAGLPKTEANW
VNVISDLKKIEDLIQSMHIDATLYTESDVHPSCKVTAMKCFLLELQVISL
ESGDASIHDTVENLIILANNSLSSNGNVTESGCKECEELEEKNIKEFLQS
FVHIVQMFINTS

SEQ ID NO: 4 is the amino acid sequence of an exemplary S309-CAR-IL-15:

MEFGLSWLFLVAILKGVQCVDQVQLVQSGAEVKKPGASVKVSCKASGYPF
TSYGISWVRQAPGQGLEWMGWISTYNGNTNYAQKFQGRVTMTTDTSTTTG
YMELRRLRSDDTAVYYCARDYTRGAWFGESLIGGFDNWGQGTLVTVSSGG
GGSGGGGSGGGGSGGGGSEIVLTQSPGTLSLSPGERATLSCRASQTVSST
SLAWYQQKPGQAPRLLIYGASSRATGIPDRFSGSGSGTDFTLTISRLEPE
DFAVYYCQQHDTSLTFGGGTKVEIKSYVTVSSQDPAEPKSPDKTHTCPPC
PAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALP
APIEKTISKAKGQPREPQVYTLPPSRDELTKNQVSLTCLVKGFYPSDIAV
EWESNGQPENNYKTTPPVLDSDGSFFLYSKLTVDKSRWQQGNVFSCSVMH
EALHNHYTQKSLSLSPGKKDPKFWVLVVVGGVLACYSLLVTVAFIIFWVR
SKRSRLLHSDYMNMTPRRPGPTRKHYQPYAPPRDFAAYRSKRGRKKLLYI
FKQPFMRPVQTTQEEDGCSCRFPEEEEGGCELRVKFSRSADAPAYQQGQN
QLYNELNLGRREEYDVLDKRRGRDPEMGGKPRRKNPQEGLYNELQKDKMA
EAYSEIGMKGERRRGKGHDGLYQGLSTATKDTYDALHMQALPPRQCTNYA
LLKLAGDVESNPGPMRISKPHLRSISIQCYLCLLLNSHFLTEAGIHVFIL
GCFSAGLPKTEANWVNVISDLKKIEDLIQSMHIDATLYTESDVHPSCKVT
AMKCFLLELQVISLESGDASIHDTVENLIILANNSLSSNGNVTESGCKEC
EELEEKNIKEFLQSFVHIVQMFINTS

SEQ ID NO: 5 is a nucleic acid sequence encoding an exemplary S309-CAR:

ATGGAGTTTGGGCTGAGCTGGCTTTTTCTTGTGGCTATTTTAAAAGGTGT
CCAGTGCGTCGACCAGGTGCAGCTGGTGCAGAGCGGCGCGGAAGTGAAAA
AACCGGGCGCGAGCGTGAAAGTGAGCTGCAAAGCGAGCGGCTATCCGTTT
ACCAGCTATGGCATTAGCTGGGTGCGCCAGGCGCCGGGCCAGGGCCTGGA
ATGGATGGGCTGGATTAGCACCTATAACGGCAACACCAACTATGCGCAGA
AATTTCAGGGCCGCGTGACCATGACCACCGATACCAGCACCACCACCGGC
TATATGGAACTGCGCCGCCTGCGCAGCGATGATACCGCGGTGTATTATTG
CGCGCGCGATTATACCCGCGGCGCGTGGTTTGGCGAAAGCCTGATTGGCG
GCTTTGATAACTGGGGCCAGGGCACCCTGGTGACCGTGAGCAGCGGTGGT
GGTGGTTCTGGTGGTGGTGGTTCTGGCGGCGGCGGCTCCGGTGGTGGTGG
ATCCGAAATTGTGCTGACCCAGAGCCCGGGCACCCTGAGCCTGAGCCCGG
GCGAACGCGCGACCCTGAGCTGCCGCGCGAGCCAGACCGTGAGCAGCACC
AGCCTGGCGTGGTATCAGCAGAAACCGGGCCAGGCGCCGCGCCTGCTGAT
TTATGGCGCGAGCAGCCGCGCGACCGGCATTCCGGATCGCTTTAGCGGCA
GCGGCAGCGGCACCGATTTTACCCTGACCATTAGCCGCCTGGAACCGGAA
GATTTTGCGGTGTATTATTGCCAGCAGCATGATACCAGCCTGACCTTTGG
CGGCGGCACCAAAGTGGAAATTAAATCGTACGTCACCGTCTCTTCACAGG
ATCCCGCCGAGCCCAAATCTCCTGACAAAACTCACACATGCCCACCGTGC
CCAGCACCTGAACTCCTGGGGGGACCGTCAGTCTTCCTCTTCCCCCCAAA
ACCCAAGGACACCCTCATGATCTCCCGGACCCCTGAGGTCACATGCGTGG
TGGTGGACGTGAGCCACGAAGACCCTGAGGTCAAGTTCAACTGGTACGTG
GACGGCGTGGAGGTGCATAATGCCAAGACAAAGCCGCGGGAGGAGCAGTA
CAACAGCACGTACCGTGTGGTCAGCGTCCTCACCGTCCTGCACCAGGACT
GGCTGAATGGCAAGGAGTACAAGTGCAAGGTCTCCAACAAAGCCCTCCCA
GCCCCCATCGAGAAAACCATCTCCAAAGCCAAAGGGCAGCCCCGAGAACC
ACAGGTGTACACCCTGCCCCCATCCCGGGATGAGCTGACCAAGAACCAGG
TCAGCCTGACCTGCCTGGTCAAAGGCTTCTATCCCAGCGACATCGCCGTG
GAGTGGGAGAGCAATGGGCAACCGGAGAACAACTACAAGACCACGCCTCC
CGTGCTGGACTCCGACGGCTCCTTCTTCCTCTACAGCAAGCTCACCGTGG
ACAAGAGCAGGTGGCAGCAGGGGAACGTCTTCTCATGCTCCGTGATGCAT
GAGGCTCTGCACAACCACTACACGCAGAAGAGCCTCTCCCTGTCTCCGGG
TAAAAAAGATCCCAAATTTTGGGTGCTGGTGGTGGTTGGTGGAGTCCTGG
CTTGCTATAGCTTGCTAGTAACAGTGGCCTTTATTATTTTTTGGGTGAGG
AGTAAGAGGAGCAGGCTCCTGCACAGTGACTACATGAACATGACTCCCCG
CCGCCCCGGGCCCACCCGCAAGCATTACCAGCCCTATGCCCCACCACGCG
ACTTCGCAGCCTATCGCTCCAAACGGGGCAGAAAGAAACTCCTGTATATA
TTCAAACAACCATTTATGAGACCAGTACAAACTACTCAAGAGGAAGATGG
CTGTAGCTGCCGATTTCCAGAAGAAGAAGAAGGAGGATGTGAACTGAGAG
TGAAGTTCAGCAGGAGCGCAGACGCCCCCGCGTACCAGCAGGGCCAGAAC
CAGCTCTATAACGAGCTCAATCTAGGACGAAGAGAGGAGTACGATGTTTT
GGACAAGAGACGTGGCCGGGACCCTGAGATGGGGGGAAAGCCGAGAAGGA
AGAACCCTCAGGAAGGCCTGTACAATGAACTGCAGAAAGATAAGATGGCG
GAGGCCTACAGTGAGATTGGGATGAAAGGCGAGCGCCGGAGGGGCAAGGG
GCACGATGGCCTTTACCAGGGTCTCAGTACAGCCACCAAGGACACCTACG
ACGCCCTTCACATGCAGGCCCTGCCCCCTCGC

SEQ ID NO: 6 is a nucleic acid encoding an exemplary E2A protein:

CAGTGTACTAATTATGCTCTCTTGAAATTGGCTGGAGATGTTGAGAGCAA
TCCCGGGCCC

SEQ ID NO: 7 is a nucleic acid encoding an exemplary IL-15 protein:

ATGCGGATCAGCAAGCCCCACCTGCGGAGCATCAGCATCCAGTGCTACCT
GTGCCTGCTGCTGAACAGCCACTTCCTGACCGAGGCCGGCATCCACGTGT
TCATCCTGGGCTGCTTCAGCGCCGGACTGCCCAAGACCGAGGCCAACTGG
GTGAACGTGATCAGCGACCTGAAGAAGATCGAGGACCTGATCCAGAGCAT
GCACATCGACGCCACCCTGTACACCGAGAGCGACGTGCACCCCAGCTGCA
AGGTGACCGCCATGAAGTGCTTTCTGCTGGAACTGCAGGTGATCAGCCTG
GAAAGCGGCGACGCCAGCATCCACGACACCGTGGAGAACCTGATCATCCT
GGCCAACAACAGCCTGAGCAGCAACGGCAACGTGACCGAGAGCGGCTGCA
AAGAGTGCGAGGAACTGGAAGAGAAGAACATCAAAGAGTTTCTGCAGAGC
TTCGTGCACATCGTGCAGATGTTCATCAACACCAGCTGA

SEQ ID NO: 8 is a nucleic acid encoding an exemplary S309-CAR-IL-15 protein:

ATGGAGTTTGGGCTGAGCTGGCTTTTTCTTGTGGCTATTTTAAAAGGTGT
CCAGTGCGTCGACCAGGTGCAGCTGGTGCAGAGCGGCGCGGAAGTGAAAA
AACCGGGCGCGAGCGTGAAAGTGAGCTGCAAAGCGAGCGGCTATCCGTTT
ACCAGCTATGGCATTAGCTGGGTGCGCCAGGCGCCGGGCCAGGGCCTGGA
ATGGATGGGCTGGATTAGCACCTATAACGGCAACACCAACTATGCGCAGA
AATTTCAGGGCCGCGTGACCATGACCACCGATACCAGCACCACCACCGGC
TATATGGAACTGCGCCGCCTGCGCAGCGATGATACCGCGGTGTATTATTG
CGCGCGCGATTATACCCGCGGCGCGTGGTTTGGCGAAAGCCTGATTGGCG
GCTTTGATAACTGGGGCCAGGGCACCCTGGTGACCGTGAGCAGCGGTGGT
GGTGGTTCTGGTGGTGGTGGTTCTGGCGGCGGCGGCTCCGGTGGTGGTGG
ATCCGAAATTGTGCTGACCCAGAGCCCGGGCACCCTGAGCCTGAGCCCGG
GCGAACGCGCGACCCTGAGCTGCCGCGCGAGCCAGACCGTGAGCAGCACC
AGCCTGGCGTGGTATCAGCAGAAACCGGGCCAGGCGCCGCGCCTGCTGAT
TTATGGCGCGAGCAGCCGCGCGACCGGCATTCCGGATCGCTTTAGCGGCA
GCGGCAGCGGCACCGATTTTACCCTGACCATTAGCCGCCTGGAACCGGAA
GATTTTGCGGTGTATTATTGCCAGCAGCATGATACCAGCCTGACCTTTGG
CGGCGGCACCAAAGTGGAAATTAAATCGTACGTCACCGTCTCTTCACAGG
ATCCCGCCGAGCCCAAATCTCCTGACAAAACTCACACATGCCCACCGTGC
CCAGCACCTGAACTCCTGGGGGGACCGTCAGTCTTCCTCTTCCCCCCAAA
ACCCAAGGACACCCTCATGATCTCCCGGACCCCTGAGGTCACATGCGTGG
TGGTGGACGTGAGCCACGAAGACCCTGAGGTCAAGTTCAACTGGTACGTG
GACGGCGTGGAGGTGCATAATGCCAAGACAAAGCCGCGGGAGGAGCAGTA
CAACAGCACGTACCGTGTGGTCAGCGTCCTCACCGTCCTGCACCAGGACT
GGCTGAATGGCAAGGAGTACAAGTGCAAGGTCTCCAACAAAGCCCTCCCA
GCCCCCATCGAGAAAACCATCTCCAAAGCCAAAGGGCAGCCCCGAGAACC
ACAGGTGTACACCCTGCCCCCATCCCGGGATGAGCTGACCAAGAACCAGG
TCAGCCTGACCTGCCTGGTCAAAGGCTTCTATCCCAGCGACATCGCCGTG
GAGTGGGAGAGCAATGGGCAACCGGAGAACAACTACAAGACCACGCCTCC
CGTGCTGGACTCCGACGGCTCCTTCTTCCTCTACAGCAAGCTCACCGTGG
ACAAGAGCAGGTGGCAGCAGGGGAACGTCTTCTCATGCTCCGTGATGCAT
GAGGCTCTGCACAACCACTACACGCAGAAGAGCCTCTCCCTGTCTCCGGG
TAAAAAAGATCCCAAATTTTGGGTGCTGGTGGTGGTTGGTGGAGTCCTGG
CTTGCTATAGCTTGCTAGTAACAGTGGCCTTTATTATTTTTTGGGTGAGG
AGTAAGAGGAGCAGGCTCCTGCACAGTGACTACATGAACATGACTCCCCG
CCGCCCCGGGCCCACCCGCAAGCATTACCAGCCCTATGCCCCACCACGCG
ACTTCGCAGCCTATCGCTCCAAACGGGGCAGAAAGAAACTCCTGTATATA
TTCAAACAACCATTTATGAGACCAGTACAAACTACTCAAGAGGAAGATGG
CTGTAGCTGCCGATTTCCAGAAGAAGAAGAAGGAGGATGTGAACTGAGAG
TGAAGTTCAGCAGGAGCGCAGACGCCCCCGCGTACCAGCAGGGCCAGAAC
CAGCTCTATAACGAGCTCAATCTAGGACGAAGAGAGGAGTACGATGTTTT
GGACAAGAGACGTGGCCGGGACCCTGAGATGGGGGGAAAGCCGAGAAGGA
AGAACCCTCAGGAAGGCCTGTACAATGAACTGCAGAAAGATAAGATGGCG
GAGGCCTACAGTGAGATTGGGATGAAAGGCGAGCGCCGGAGGGGCAAGGG
GCACGATGGCCTTTACCAGGGTCTCAGTACAGCCACCAAGGACACCTACG
ACGCCCTTCACATGCAGGCCCTGCCCCCTCGCCAGTGTACTAATTATGCT
CTCTTGAAATTGGCTGGAGATGTTGAGAGCAATCCCGGGCCCATGCGGAT
CAGCAAGCCCCACCTGCGGAGCATCAGCATCCAGTGCTACCTGTGCCTGC
TGCTGAACAGCCACTTCCTGACCGAGGCCGGCATCCACGTGTTCATCCTG
GGCTGCTTCAGCGCCGGACTGCCCAAGACCGAGGCCAACTGGGTGAACGT
GATCAGCGACCTGAAGAAGATCGAGGACCTGATCCAGAGCATGCACATCG
ACGCCACCCTGTACACCGAGAGCGACGTGCACCCCAGCTGCAAGGTGACC
GCCATGAAGTGCTTTCTGCTGGAACTGCAGGTGATCAGCCTGGAAAGCGG
CGACGCCAGCATCCACGACACCGTGGAGAACCTGATCATCCTGGCCAACA
ACAGCCTGAGCAGCAACGGCAACGTGACCGAGAGCGGCTGCAAAGAGTGC
GAGGAACTGGAAGAGAAGAACATCAAAGAGTTTCTGCAGAGCTTCGTGCA
CATCGTGCAGATGTTCATCAACACCAGCTGA

SEQ ID NOs: 9 and 10 are SARS-COV-2 S gene forward and reverse primers, respectively.

SEQ ID NOs: 11-22 are primer sequences for subcloning S protein variants.

### DETAILED DESCRIPTION

Recent clinical trials testing cancer immunotherapies have shown promising results for treating infectious diseases. As demonstrated herein, S309-CAR-NK-92MI, a NK-92 cell line capable of producing the IL-2 molecule to sustain its own persistence in vivo, provides a proof-of-concept for using S309-CAR-based cell therapy for treating severe COVID-19 patients. These results were confirmed by using primary NK cells expanded from peripheral blood. The disclosed CARs also demonstrate improved results compared to previous CARs targeting coronavirus spike protein. These experiments provide the basis for additional preclinical studies and a potential clinical application for treating coronavirus infections.

### I. Terms

Unless otherwise noted, technical terms are used according to conventional usage. Definitions of common terms in molecular biology may be found in Lewin's Genes X, ed. Krebs et al., Jones and Bartlett Publishers, 2009 (ISBN 0763766321); Kendrew et al. (eds.), The Encyclopedia of Molecular Biology, published by Blackwell Publishers, 1994 (ISBN 0632021829); Robert A. Meyers (ed.), Molecular Biology and Biotechnology: a Comprehensive Desk Reference, published by Wiley, John & Sons, Inc., 1995 (ISBN 0471186341); and George P. Rédei, Encyclopedic Dictionary of Genetics, Genomics, Proteomics and Informatics, 3^{rd} Edition, Springer, 2008 (ISBN: 1402067534), and other similar references.

Unless otherwise explained, all technical and scientific terms used herein have the same meaning as commonly understood by one of ordinary skill in the art to which this disclosure belongs. The singular terms “a,” “an,” and “the” include plural referents unless the context clearly indicates otherwise. “Comprising A or B” means including A, or B, or A and B. It is further to be understood that all base sizes or amino acid sizes, and all molecular weight or molecular mass values, given for nucleic acids or polypeptides are approximate, and are provided for description.

Although methods and materials similar or equivalent to those described herein can be used in the practice or testing of the present disclosure, suitable methods and materials are described below. All publications, patent applications, patents, and other references mentioned herein are incorporated by reference in their entirety, as are the GenBank or Protein Databank Accession numbers (as present in the database on Dec. 15, 2020). In case of conflict, the present specification, including explanations of terms, will control. In addition, the materials, methods, and examples are illustrative only and not intended to be limiting.

In order to facilitate review of the various embodiments of the disclosure, the following explanations of specific terms are provided:

Antibody: A polypeptide ligand comprising at least one variable region that recognizes and binds (such as specifically recognizes and specifically binds) an epitope of an antigen. Mammalian immunoglobulin molecules are composed of a heavy (H) chain and a light (L) chain, each of which has a variable region, termed the variable heavy (V_{H}) region and the variable light (V_{L}) region, respectively. Together, the VH region and the VL region are responsible for binding the antigen recognized by the antibody. There are five main heavy chain classes (or isotypes) of mammalian immunoglobulin, which determine the functional activity of an antibody molecule: IgM, IgD, IgG, IgA and IgE.

Antibody variable regions contain “framework” regions and hypervariable regions, known as “complementarity determining regions” or “CDRs.” The CDRs are primarily responsible for binding to an epitope of an antigen. The framework regions of an antibody serve to position and align the CDRs in three-dimensional space. The amino acid sequence boundaries of a given CDR can be readily determined using any of a number of well-known numbering schemes, including those described by Kabat et al. (Sequences of Proteins of Immunological Interest, U.S. Department of Health and Human Services, 1991; the “Kabat” numbering scheme), Chothia et al. (see Chothia and Lesk, J Mol Biol 196:901-917, 1987; Chothia et al., Nature 342:877, 1989; and Al-Lazikani et al., (JMB 273,927-948, 1997; the “Chothia” numbering scheme), and the ImMunoGeneTics (IMGT) database (see, Lefranc, Nucleic Acids Res 29:207-9, 2001; the “IMGT” numbering scheme). The Kabat and IMGT databases are maintained online.

A single-chain antibody (scFv) is a genetically engineered molecule containing the V_{H} and V_{L} domains of one or more antibody(ies) linked by a suitable polypeptide linker as a genetically fused single chain molecule (see, for example, Bird et al., Science, 242:423-426, 1988; Huston et al., Proc. Natl. Acad. Sci., 85:5879-5883, 1988; Ahmad et al., Clin. Dev. Immunol., 2012, doi: 10.1155/2012/980250; Marbry, IDrugs, 13:543-549, 2010). The intramolecular orientation of the V_{H}-domain and the V_{L}-domain in a scFv, is typically not decisive for scFvs. Thus, scFvs with both possible arrangements (V_{H}-domain-linker domain-V_{L}-domain; V_{L}-domain-linker domain-V_{H}-domain) may be used. In a dsFv the V_{H} and V_{L} have been mutated to introduce a disulfide bond to stabilize the association of the chains. Diabodies also are included, which are bivalent, bispecific antibodies in which V_{H} and V_{L} domains are expressed on a single polypeptide chain, but using a linker that is too short to allow for pairing between the two domains on the same chain, thereby forcing the domains to pair with complementary domains of another chain and creating two antigen binding sites (see, for example, Holliger et al., Proc. Natl. Acad. Sci., 90:6444-6448, 1993; Poljak et al., Structure, 2:1121-1123, 1994).

Antibodies also include genetically engineered forms such as chimeric antibodies (such as humanized murine antibodies) and heteroconjugate antibodies (such as bispecific antibodies). See also, Pierce Catalog and Handbook, 1994-1995 (Pierce Chemical Co., Rockford, IL); Kuby, J., Immunology, 3^{rd} Ed., W.H. Freeman & Co., New York, 1997.

Chimeric antigen receptor (CAR): A chimeric molecule that includes an antigen-binding portion (such as a single domain antibody or scFv) and a signaling domain, such as a signaling domain from a T cell receptor (e.g. CD3ζ). Typically, CARs include an antigen-binding portion, a transmembrane domain, and an intracellular domain. The intracellular domain typically includes a signaling domain having an immunoreceptor tyrosine-based activation motif (ITAM), such as CD3ζ or FcεRIγ. In some instances, the intracellular domain also includes the intracellular portion of at least one additional co-stimulatory domain, such as a co-stimulatory domain from CD28, 4-1BB (CD137), ICOS, OX40 (CD134), CD27, and/or DAP10.

Complementarity determining region (CDR): A region of hypervariable amino acid sequence that defines the binding affinity and specificity of an antibody. The light and heavy chains of a mammalian immunoglobulin each have three CDRs, designated VL-CDR1, VL-CDR2, VL-CDR3 and VH-CDR1, VH-CDR2, VH-CDR3, respectively.

Coronavirus: Coronaviruses are a large family of positive-sense, single-stranded RNA viruses that can infect humans and non-human animals. The viral envelope is composed of a lipid bilayer containing the viral membrane (M), envelope (E) and spike(S) proteins. Most coronaviruses cause mild to moderate upper respiratory tract illness; however, three coronaviruses have emerged that can cause more serious illness and death in humans. These are two severe acute respiratory syndrome coronaviruses (SARS-COV and SARS-COV-2) and Middle East respiratory syndrome coronavirus (MERS-COV). Other coronaviruses that infect humans include human coronavirus HKU1, human coronavirus OC43, human coronavirus 229E, and human coronavirus NL63.

Isolated: An “isolated” biological component, such as a nucleic acid, protein, or organelle, has been substantially separated or purified away from other biological components in the environment (such as a cell) in which the component occurs, e.g., other chromosomal and extra-chromosomal DNA and RNA, proteins, and organelles. Nucleic acids and proteins that have been “isolated” include nucleic acids and proteins purified by standard purification methods. The term also embraces nucleic acids and proteins prepared by recombinant expression in a host cell as well as chemically synthesized nucleic acids and proteins.

Natural Killer (NK) cells: Cells of the immune system that kill target cells in the absence of a specific antigenic stimulus and without restriction according to MHC class. Target cells can be tumor cells or cells harboring viruses. NK cells are characterized by the presence of CD56 and the absence of CD3 surface markers. NK cells typically comprise approximately 10 to 15% of the mononuclear cell fraction in normal peripheral blood. Historically, NK cells were first identified by their ability to lyse certain tumor cells without prior immunization or activation. NK cells are thought to provide a “back up” protective mechanism against viruses and tumors that might escape the CTL response by down-regulating MHC class I presentation. In addition to being involved in direct cytotoxic killing, NK cells also serve a role in cytokine production, which can be important to control cancer and infection.

In some examples, a “modified NK cell” is a NK cell transduced or transfected with a heterologous nucleic acid (such as one or more of the nucleic acids or vectors disclosed herein) or expressing one or more heterologous proteins (such as one or more CARs disclosed herein). The terms “modified NK cell” and “transduced NK cell” are used interchangeably in some examples herein.

Pharmaceutically acceptable carriers: Remington: The Science and Practice of Pharmacy, 22^{nd} ed., London, UK: Pharmaceutical Press (2013), describes compositions and formulations suitable for pharmaceutical delivery of modified immune cells and other compositions disclosed herein. In general, the nature of the carrier will depend on the particular mode of administration being employed. For instance, parenteral formulations usually include pharmaceutically and physiologically acceptable fluids such as water, physiological saline, balanced salt solutions, aqueous dextrose, glycerol, or the like as a vehicle. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Purified: The term purified does not require absolute purity; rather, it is intended as a relative term. Thus, for example, a purified protein, nucleic acid, or cell preparation is one in which the protein, nucleic acid, or cell is more enriched than in its initial environment. In one embodiment, a preparation is purified such that the protein, nucleic acid, or cell represents at least 50% of the total protein, nucleic acid, or cell content of the preparation. Substantial purification denotes purification from other proteins, nucleic acids, or cells. A substantially purified protein, nucleic acid, or cell is at least 60%, 70%, 80%, 90%, 95%, 98%, or 99% pure. Thus, in one specific, non-limiting example, a substantially purified protein, nucleic acid, or cell is 90% free of other components.

Recombinant: A nucleic acid or protein that has a sequence that is not naturally occurring or has a sequence that is made by an artificial combination of two otherwise separated segments of sequence (e.g., a “chimeric” sequence). This artificial combination can be accomplished by chemical synthesis or by the artificial manipulation of isolated segments of nucleic acids, for example, by genetic engineering techniques.

SARS-COV-2: A virus of the genus betacoronavirus that first emerged in humans in 2019, also referred to as Wuhan coronavirus, 2019-nCOV, or 2019 novel coronavirus. Symptoms of SARS-COV-2 infection include fever, chills, dry cough, shortness of breath, fatigue, muscle/body aches, headache, new loss of taste or smell, sore throat, nausea or vomiting, and diarrhea. Patients with severe disease can develop pneumonia, multi-organ failure, and death. The SARS-COV-2 virion includes a viral envelope with large spike glycoproteins. The SARS-COV-2 genome encodes a canonical set of structural protein genes in the order 5′-spike(S)-envelope (E)-membrane (M)-nucleocapsid (N)-3′.

Spike(S) protein: A class I fusion glycoprotein initially synthesized as a precursor protein of approximately 1256 amino acids for SARS-COV, and 1273 amino acids for SARS-COV-2. Individual precursor S polypeptides form a homotrimer and undergo glycosylation and processing to remove the signal peptide, and cleavage by a cellular protease between approximately position 679/680 for SARS-COV, and 685/686for SARS-COV-2, to generate separate S1 and S2 polypeptide chains, which remain associated as S1/S2 protomers within the homotrimer, thereby forming a trimer of heterodimers. The SI subunit is distal to the virus membrane and contains the receptor-binding domain (RBD) that is believed to mediate virus attachment to its host receptor. The S2 subunit is believed to contain the fusion protein machinery, such as the fusion peptide. S2 also includes two heptad-repeat sequences (HR1 and HR2) and a central helix typical of fusion glycoproteins, a transmembrane domain, and a cytosolic tail domain. An exemplary SARS-COV-2 spike protein sequence includes GenBank Accession No. QHD43416.1 (the sequence of which is incorporated by reference herein).

Subject: A living multi-cellular vertebrate organism, a category that includes both human and veterinary subjects, including human and non-human mammals. T cell: A white blood cell (lymphocyte) that is an important mediator of the immune response. T cells include, but are not limited to, CD4⁺ T cells and CD8⁺ T cells. A CD4⁺ T lymphocyte is an immune cell that carries a marker on its surface known as “cluster of differentiation 4” (CD4). These cells, also known as helper T cells, help orchestrate the immune response, including antibody responses as well as killer T cell responses. CD8⁺ T cells carry the “cluster of differentiation 8” (CD8) marker. In one embodiment, a CD8⁺ T cell is a cytotoxic T lymphocyte (CTL). In another embodiment, a CD8⁺ cell is a suppressor T cell.

Activated T cells can be detected by an increase in cell proliferation and/or expression of or secretion of one or more cytokines (such as IL-2, IL-4, IL-6, IFNγ, or TNFα). Activation of CD8⁺ T cells can also be detected by an increase in cytolytic activity in response to an antigen.

In some examples, a “modified T cell” is a T cell transduced or transfected with a heterologous nucleic acid (such as one or more of the nucleic acids or vectors disclosed herein) or expressing one or more heterologous proteins (such as one or more CARs disclosed herein). The terms “modified T cell” and “transduced T cell” are used interchangeably in some examples herein.

Transduced or Transformed: A transformed cell is a cell into which a nucleic acid molecule has been introduced by molecular biology techniques. As used herein, the terms transduction and transformation encompass all techniques by which a nucleic acid molecule might be introduced into such a cell, including transfection with viral vectors, the use of plasmid vectors, and introduction of DNA by electroporation, lipofection, and particle gun acceleration.

Treating or ameliorating a disease: “Treating” refers to a therapeutic intervention that decreases or inhibits a sign or symptom of a disease or pathological condition after it has begun to develop “Ameliorating” refers to the reduction in the number or severity of signs or symptoms of a disease, such as that caused by SARS coronaviruses.

Vector: A nucleic acid molecule that can be introduced into a host cell (for example, by transfection or transduction), thereby producing a transformed host cell. Recombinant DNA vectors are vectors having recombinant DNA. A vector can include nucleic acid sequences that permit it to replicate in a host cell, such as an origin of replication. A vector can also include one or more selectable marker genes and other genetic elements known in the art. Viral vectors are recombinant nucleic acid vectors having at least some nucleic acid sequences derived from one or more viruses. A replication deficient viral vector is a vector that requires complementation of one or more regions of the viral genome required for replication due to a deficiency in at least one replication-essential gene function.

### II. S309-CAR and S309-CAR Cells

Provided herein are CARs that include a coronavirus spike protein-specific binding portion. In some embodiments, the CAR includes an antigen binding domain including a S309 antibody scFv, a hinge domain, a transmembrane domain, and an intracellular domain. In additional embodiments, the CAR further includes a signal peptide and/or an interleukin-15 domain.

In some embodiments, the antigen binding domain is a coronavirus spike protein-specific scFv from S309 antibody, for example having an amino acid sequence with at least 90% sequence identity (for example, at least 91%, at least 92%, at least 93%, at least 94%, at least 95%, at least 96%, at least 97%, at least 98% at least 99% identity) to amino acids 22-275 of SEQ ID NO: 1 or including or consisting of amino acids 22-275 of SEQ ID NO: 1. In other embodiments, the antigen binding domain includes at least one of the CDR sequences (e.g., at least one of VHCDR1-3 and VLCDR1-3, such as at least 1, 2, 3, 4, 5, or 6 of the CDR sequences) provided in Table 1, and specifically binds to a coronavirus spike protein. In some embodiments, the antigen binding domain includes the variable heavy chain (VH) domain complementarity determining region 1 (CDR1), CDR2 and CDR3 amino acid sequences of amino acid positions 47-54, 72-79, and 118-137 of SEQ ID NO: 1, respectively, and the variable light chain (VL) domain CDR1, CDR2 and CDR3 amino acid sequences of amino acid positions 195-201, 219-221, and 258-265 of SEQ ID NO: 1, respectively.

TABLE 1
Location of the CDRs in the S309 scFv sequence (determined using IGMT
method)

| CDR | Nucleic Acid Sequence | Amino Acid Sequence |
|---|---|---|
| VH CDR1 | GGCTATCCGTTTACCAGCTATGGC | GYPFTSYG |
| | (139-162 of SEQ ID NO: 5) | (47-54 of SEQ ID NO: 1) |
| VH CDR2 | ATTAGCACCTATAACGGCAACACC | ISTYNGNT |
| | (214-237 of SEQ ID NO: 5) | (72-79 of SEQ ID NO: 1) |
| VH CDR3 | GCGCGCGATTATACCCGCGGCGCGTGGT | ARDYTRGAWFGESLIGG |
| | TTGGCGAAAGCCTGATTGGCGGCTTTGA | FDN |
| | TAAC (352-411 of SEQ ID NO: 5) | (118-137 of SEQ ID NO: 1) |
| VL CDR1 | CAGACCGTGAGCAGCACCAGC | QTVSSTS |
| | (583-603 of SEQ ID NO: 5) | (195-201 of SEQ ID NO: 1) |
| VL CDR2 | GGCGCGAGC | GAS |
| | (655-663 of SEQ ID NO: 5) | (219-221 of SEQ ID NO: 1) |
| VL CDR3 | CAGCAGCATGATACCAGCCTGACC | QQHDTSLT |
| | (772-795 of SEQ ID NO: 5) | (258-265 OF SEQ ID NO: 1) |

In some embodiments, the hinge domain is an IgG hinge region. In one example, the hinge domain is an IgG1 hinge. Other hinge domains can be used, such as hinge regions from other immunoglobulins (for example, IgG4 or IgD) or a hinge region from CD8, CD28, or CD40. In particular examples, the hinge domain includes amino acids 287-518 of SEQ ID NO: 1.

In additional embodiments, the transmembrane domain is a CD28 transmembrane domain. In one example, the transmembrane domain is from CD28. The transmembrane domain can also be from other proteins, such as CD8, CD4, CD35., CD40, OX40L, 41BBL, ICOS, ICOS-L, CD80, CD86, ICAM-1, LFA-1, ICAM-1, CD56, CTLA-4, PD-1, TIM-3, NKP30, NKP44, NKP40, NKP46, B7-H3, PD-L1, PD-2, and CD70. In some examples, the CD28 transmembrane domain includes amino acids 523-549 of SEQ ID NO: 1.

In further embodiments, the intracellular domain includes one or more intracellular regions from CD28, 4-1BB, and/or CD3ζ. In particular examples, the intracellular domain includes domains from CD28, 4-1BB, and CD35. In one non-limiting example, the intracellular domain includes amino acids 550-774 of SEQ ID NO: 1. Other exemplary intracellular regions that can be included are from CD8, CD40, OX-40, ICOS, CD27, DAP10, OX40-L, 4-1BBL, ICOS-L, CD80, CD86, ICAM-1, LFA-1, CD56, CTLA-4, PD-1, TIM-3, NKP30, NKP44, NKP40, NKP46, B7-H3, PD-L1, PD-2, CD70, DAP12, PDK, or FcεRIγ.

In some embodiments, the S309-CAR also includes a signal peptide, which is located N-terminal to the scFv domain. In some examples, the signal sequence is a IgG signal peptide or a GM-CSF signal peptide. In one example, the signal sequence is amino acids 1-19 of SEQ ID NO: 1.

In other embodiments, the S309-CAR further includes a domain that increases survival or persistence of a modified immune cell expressing the CAR. In some examples, the domain is a cytokine, for example, an interleukin (IL), such as IL-15 (e.g., SEQ ID NO: 3) or IL-12. In some examples, the domain is located C-terminal to the CD32 domain of the CAR.

In additional embodiments, the CAR further includes an inducible gene that can be used to eliminate CAR expressing cells (e.g., a “suicide” gene). The inducible gene can be activated in the event of off target side effects, such as cytokine release syndrome (“cytokine storm”). In some examples, expression of the suicide gene is inducible by a small molecule, such as tetracycline or doxycycline (a “TET ON” system) or rapamycin. See, e.g., Gargett et al., Front. Pharmacol. 5:235, 2014; Stavrou et al., Mol. Ther. 6:1266-1276, 2018. In other examples, the suicide gene is inducible by a Fas domain inducible system. In some examples, the inducible suicide domain is located N-terminal or C-terminal to the antigen binding domain of the CAR, while in other examples, the inducible suicide domain is located C-terminal to the CD33 domain of the CAR. The inducible suicide domain is separated from the CAR by a self-cleaving peptides (such as a P2A peptide or T2A peptide).

In some embodiments, the disclosed CARs further include one or more additional antigen binding domains (for example, the CAR is a bispecific CAR or a tri-specific CAR), which may be N-terminal or C-terminal to the S309 antigen binding domain. In some examples, the CAR includes at least one additional antigen binding domain that specifically binds to a coronavirus spike protein or a variant thereof. In some examples, the additional antigen binding domain is different from the S309 antigen binding domain. In some examples, the additional antigen binding domain specifically binds to a coronavirus spike protein including a D614G amino acid substitution. One of ordinary skill in the art can select D614G antigen binding domains (see, e.g., Cao et al., bioRxiv, doi.org/10.1101/2020.09.27.316174, 2020). In other examples, the additional antigen binding domain specifically binds to a coronavirus spike protein from coronavirus lineage B.1.617.2, AY.1, AY.2, or AY.3 (e.g., “delta” variant coronaviruses) or a variant thereof. In other examples, the additional antigen binding domain specifically binds to a coronavirus envelope protein or a variant thereof or a coronavirus membrane protein or a variant thereof. In some embodiments, one or more of the additional antigen binding domains specifically binds to a SARS-COV-2 protein (such as a spike, envelope, or membrane protein). Any combination of additional antigen binding domains can be included in the CAR, such as one or more antigen binding domains that specifically bind a coronavirus spike protein, one or more antigen binding domains that specifically bind a coronavirus envelope protein, one or more antigen binding domains that specifically bind a coronavirus membrane protein, or any combination thereof. In one example, the CAR includes the S309 antigen binding domain disclosed herein and a second antigen binding domain that specifically binds to a coronavirus spike protein and is different from the S309 antigen binding domain, for example is a CR3022 antigen binding domain (see, e.g., U.S. application Ser. No. 17/399,993, filed Aug. 11, 2021, which is incorporated herein by reference in its entirety).

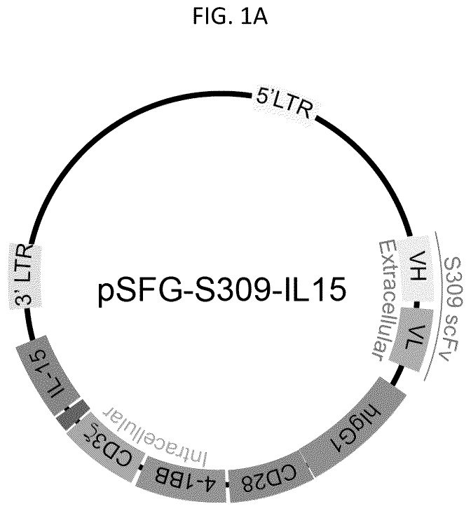

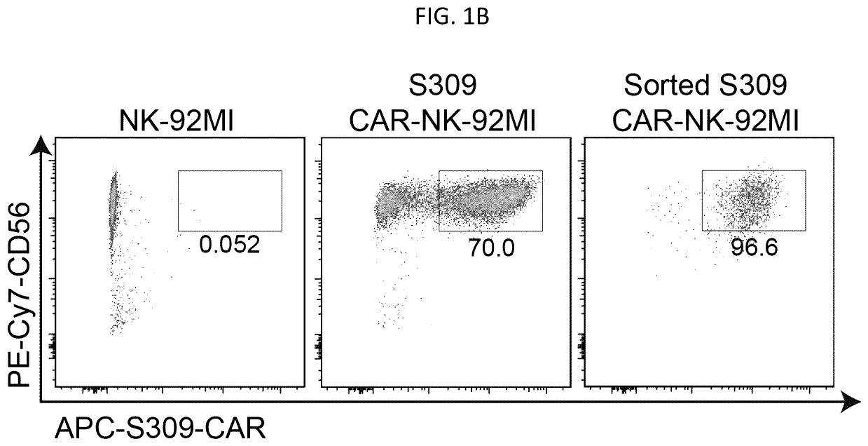

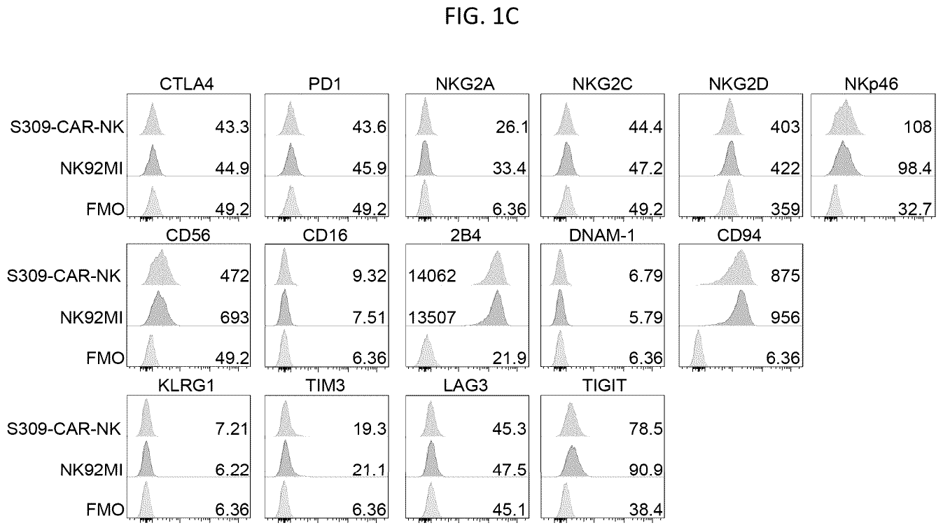

An exemplary S309-CAR is illustrated in FIG. 1A. In one example, the S309-CAR has an amino acid sequence with at least 90% sequence identity (for example, at least 91%, at least 92%, at least 93%, at least 94%, at least 95%, at least 96%, at least 97%, at least 98% or at least 99% sequence identity) to SEQ ID NO: 1 or SEQ ID NO: 4. In other examples, the S309-CAR includes or consists of the amino acid sequence of SEQ ID NO: 1 or SEQ ID NO: 4.

Also provided are nucleic acids encoding the S309 scFv and S309-CARs disclosed herein. In some examples, the S309 scFV or the antigen binding domain of the CAR is encoded by a nucleic acid including the variable heavy chain (VH) domain complementarity determining region 1 (CDR1), CDR2 and CDR3 nucleic acid sequences of nucleic acid positions 139-162, 514-237, and 352-411 of SEQ ID NO: 5,respectively, and the variable light chain (VL) domain CDR1, CDR2 and CDR3 nucleic acid sequences of nucleic acid positions 583-603, 655-663, and 772-765 of SEQ ID NO: 5, respectively. In particular examples, the S309 scFv encoding sequence is a codon-optimized sequence. In some examples, the S309 scFv is encoded by a nucleic acid sequence with at least 90% sequence identity (for example, at least 91%, at least 92%, at least 93%, at least 94%, at least 95%, at least 96%, at least 97%, at least 98% or at least 99% sequence identity) to nucleotides 58-825 of SEQ ID NO: 5 or includes or consists of nucleotides 58-825 of SEQ ID NO: 5. In other examples, the S309-CAR is encoded by a nucleic acid sequence with at least 90% sequence identity (for example, at least 91%, at least 92%, at least 93%, at least 94%, at least 95%, at least 96%, at least 97%, at least 98% or at least 99% sequence identity) to SEQ ID NO: 5 or SEQ ID NO: 8 or includes or consists of the nucleic acid sequence of SEQ ID NO: 5 or SEQ ID NO: 8.

In some embodiments, a nucleic acid molecule encoding a disclosed CAR is included in an vector (such as a viral vector) for expression in a host cell, such as a NK cell. In some examples, the expression vector includes a promoter operably linked to the nucleic acid molecule encoding the CAR. Additional expression control sequences, such as one or more enhancers, transcription and/or translation terminators, and initiation sequences can also be included in the expression vector. In some embodiments, a nucleic acid encoding a CAR provided herein is included in a viral vector. Examples of suitable virus vectors include retrovirus (e.g., MoMLV or lentivirus), adenovirus, adeno-associated virus, vaccinia virus, and fowlpox vectors. In specific examples, the CAR-encoding nucleic acid is included in a MoMLV vector, such as an SFG retroviral vector, or a pHAGE-CPPT lentiviral vector. In other examples, the vector may be a DNA vector.

In some examples, the vector further includes a nucleic acid sequence encoding at least one additional CAR. In some examples, the additional CAR is specific to a coronavirus antigen, for example, a coronavirus spike protein. In some examples, the one or more additional CARs are included in the vector with a CAR disclosed herein, for example, separated by a self-cleaving peptide, such as a P2A peptide sequence. In one example, the additional CAR is a CR3022-CAR (sec, e.g., U.S. application Ser. No. 17/399,993, filed Aug. 11, 2021, which is incorporated herein by reference in its entirety)

Also provided herein are cells (for example, immune cells) that express the disclosed CARs. In particular embodiments, the cells include NK cells. In one non-limiting embodiment, the cell is an NK-92 cell. NK-92 cells are a NK cell line derived from a patient with non-Hodgkin's lymphoma (e.g., ATCC® CRL-2407™). This cell line has properties of activated NK cells (see, e.g., Gong et al., Leukemia 8:652-658, 1994). In another embodiment, the cell is an NK-92MI cell (e.g., ATCC® CRL-2408™). The NK-92MI cell line is an interleukin-2 (IL-2) independent NK cell line, derived from NK-92, which stably expresses human IL-2 (see, e.g., Tam et al., Hum. Gene Ther. 10:1359-1373, 1999). NK-92 or NK-92MI cells expressing a CAR (such as a S309-CAR and/or other nucleic acids disclosed herein) can be used herein as an “off the shelf” immunotherapy, since autologous NK cells do not have to be produced for each subject. Other NK cell lines that can be used with the disclosed CARs described herein include NKL, KHYG-1, and YTS cells. In other embodiments, the cells include T cells, NKT cells, or macrophages.

Commonly, NK-92 cells must be irradiated prior to infusion to prevent permanent engraftment. The amount of irradiation required is around 10 Gy. The dose of irradiated NK-92 infusion can be up to 10¹⁰ NK-92 cells/m². Importantly, irradiated NK-92 cells have been shown to be safe for infusion in patients, as demonstrated by several NK-92 clinical trials (NCT00900809, NCT00990717, NCT00995137, and NCT01974479). In some examples, the cells are irradiated following transduction or transfection (e.g., treated with γ-irradiation, such as at a dose of at least 1,000, at least 2,000, at least 3,000, at least 5,000, at least 7,000, at least 8,000, at least 9,000, at least 10,000, at least 11,000, at least 12,000, or at least 15,000 or about 1,000-15,000, 2,000-12,000, 1,000-5,000, 5,000-10,000, or 8,000-12,000, or about 10,000 Rad), for example, prior to administering to a subject.

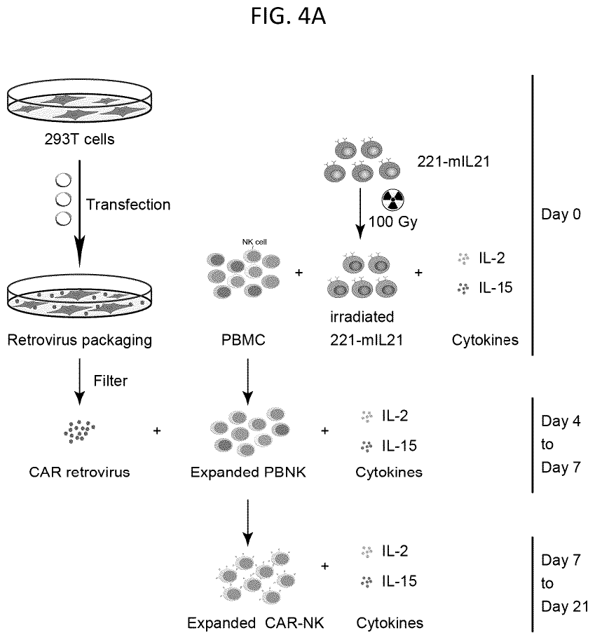

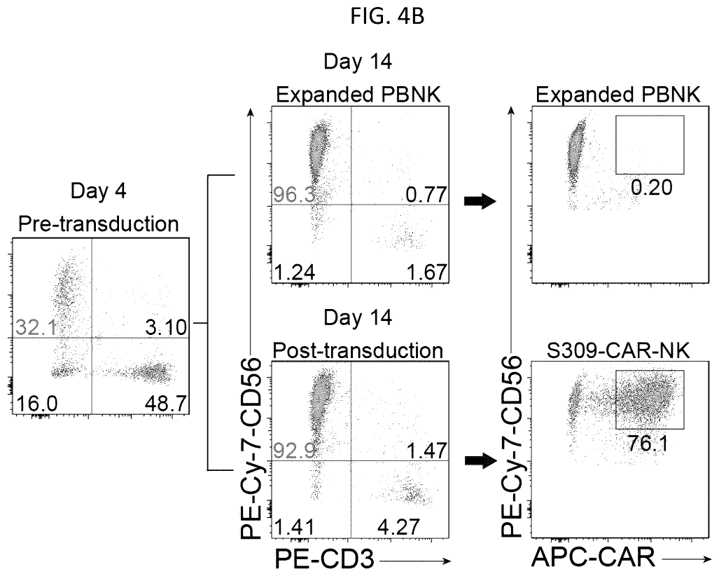

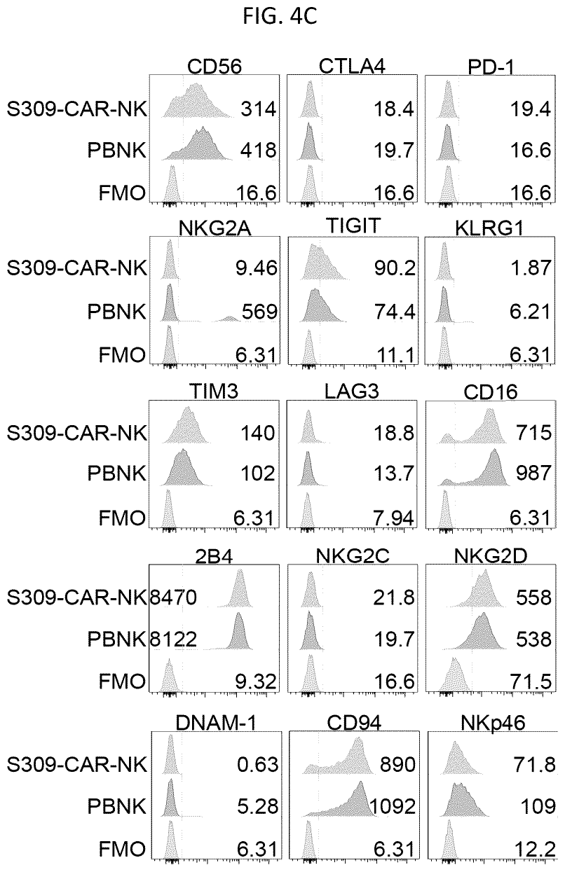

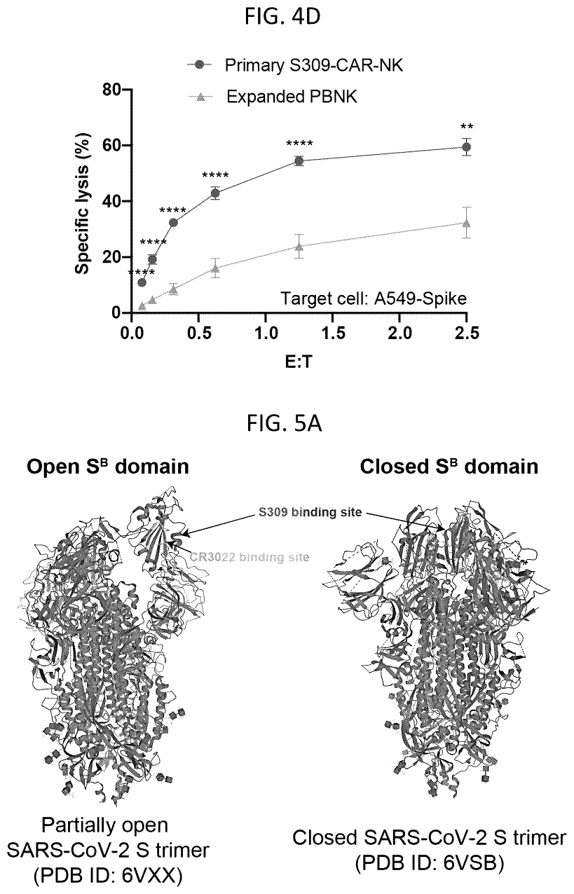

In some non-limiting embodiments, immune cells (such as NK cells or T cells) are transduced with a vector or virus encoding a S309-CAR, including but not limited to SEQ ID NOs: 5 and 8 provided herein. Following transduction, cells expressing the S309-CAR can be detected and/or enriched, for example, by flow cytometry using a labeled antibody that binds to SARS spike protein. In some examples, the transduced cells are expanded, for example, by cell culture for a period of time following transduction. In some examples, some or all of the modified cells are cryopreserved for later use. An exemplary method of producing CAR-NK cells is illustrated in FIG. 4A. However, one of ordinary skill in the art will understand that additional methods of preparing CAR-NK cells can also be successfully utilized. Methods of preparing CAR-T cells are also known.

### III. Methods of Treating Coronavirus

Provided herein are methods of treating coronavirus infection (such as SARS-CoV or SARS-COV2 infection) in a subject using a S309-CAR disclosed herein. In some embodiments, the methods include administering to the subject a composition including a modified cell (such as a modified NK cell) expressing a S309-CAR (for example, transduced with a virus or vector encoding the CAR) and a pharmaceutically acceptable carrier. In other examples, the methods include administering to the subject a pharmaceutical composition including an expression vector encoding a S309-CAR and a pharmaceutically acceptable carrier. In some examples, the subject has been identified as being infected with a coronavirus or is suspected of being infected with a coronavirus. In particular examples, the coronavirus is SARS-COV-2.

In additional embodiments, the subject may be administered an additional therapeutic agent, for example, modified immune cells expressing a second CAR, which in one non-limiting example is a CAR that specifically binds to D614G mutant coronavirus spike protein. The second CAR may be expressed in the same cells as the S309 CAR disclosed herein, or may be expressed in different cells.

The modified cells or nucleic acids expressing a S309-CAR described herein can be incorporated into pharmaceutical compositions. In some examples, the compositions include a population of cells (such as S309-CAR-NK cells) and a pharmaceutically acceptable carrier. A “pharmaceutically acceptable carrier” includes any and all solvents, dispersion media, coatings, antibacterial and antifungal agents, isotonic and absorption delaying agents, and the like, compatible with pharmaceutical administration (sec, e.g., Remington: The Science and Practice of Pharmacy, 22^{nd} ed., London, UK: Pharmaceutical Press, 2013). Examples of such carriers or diluents include, but are not limited to, water, saline, Ringer's solutions, dextrose solution, balanced salt solutions, and/or 5% human serum albumin. Liposomes and non-aqueous vehicles such as fixed oils may also be used. Supplementary active compounds can also be incorporated into the compositions. Actual methods for preparing administrable compositions include those provided in Remington: The Science and Practice of Pharmacy, 22^{nd} ed., London, UK, Pharmaceutical Press, 2013.

In some examples, the subject being treated is infected with or is suspected to be infected with a coronavirus (such as SARS-COV or SARS-COV2). In particular examples, the subject has COVID-19 disease, caused by infection with SARS-COV2. The population of modified cells (such as S309-CAR-NK cells) is typically administered parenterally, for example intravenously; however, other routes of administration can be utilized. Appropriate routes of administration can be determined based on factors such as the subject, the condition being treated, and other factors.

In some examples, the composition includes about 10⁴ to 10¹² modified immune cells (for example, about 10⁴-10⁸ cells, about 10⁶-10⁸ cells, or about 10⁶-10¹² cells). For example, the composition may be prepared such that about 10⁴ to 10¹⁰ modified cells/kg (such as about 10⁴, 10⁵, 10⁶, 10⁷, or 10⁸ cells/kg) are administered to a subject. In specific examples, the composition includes at least 10⁴, 10⁵, 10⁶, or 10⁷ S309-CAR cells (such as S309-CAR-NK cells). Multiple doses of the population of modified cells can be administered to a subject. For example, S309-CAR cells can be administered daily, every other day, twice per week, weekly, every other week, every three weeks, monthly, or less frequently. A skilled clinician can select an administration schedule based on the subject, the condition being treated, the previous treatment history, and other factors.

In additional examples, the subject is also administered at least one, at least two, at least three, or at least four cytokine(s) (such as IL-2, IL-15, IL-21, and/or IL-12) to support survival and/or growth of the modified immune cells. In specific, non-limiting examples, at least one cytokine includes IL-2 and IL-15. The cytokine(s) are administered before, after, or substantially simultaneously with the modified cells. In specific examples, at least one cytokine (e.g., IL-2) is administered simultaneously with the S309-CAR cells, for example, with S309-CAR-NK cells.

### EXAMPLES

The following examples are provided to illustrate certain particular features and/or embodiments. These examples should not be construed to limit the disclosure to the particular features or embodiments described.

### Example 1

### Materials and Methods

Antibodies and Reagents: PE anti-human CD3 antibody (clone OKT3), FITC and PE/Cy7 anti-human CD56 antibody (clone HCD56, BioLegend), PE anti-human CD69 antibody (clone FN50, BioLegend), PE anti-human CD8a antibody (clone RPA-T8, BioLegend), APC/Fire 750 anti-human CD226 antibody (DNAM-1) (clone 11A8, BioLegend), APC/Fire 750 anti-human KLRG1 (MAFA) antibody (clone SA231A2, BioLegend), BV421 anti-human CD335 (NKp46) antibody (clone 9E2, BioLegend), PE/Cy7 anti-human CD244 (2B4) antibody (clone C1.7, BioLegend), PE anti-human CD152 (CTLA-4) antibody (clone BNI3), APC anti-human CD366 (Tim-3) antibody (clone F38-2E2), PerCP/Cy5.5 anti-human TIGIT (VSTM3) antibody (clone A15153G), FITC anti-human CD223 (LAG-3) antibody (clone 11C3C65, BioLegend), BV510 anti-human CD314 (NKG2D) antibody (clone 1D11), and APC anti-human CD94 (clone DX22, BioLegend) were purchased from BioLegend (San Diego, CA, USA). APC anti-human CD16 antibody (clone 3G8, BD Biosciences), BV711 anti-human CD314 (NKG2D) antibody (clone 1D11, BD Biosciences), and FITC anti-human CD107a antibody (clone H4A3, BD Biosciences) were purchased from BD Biosciences (San Jose, CA, USA). PE anti-human NKG2C/CD159c antibody (clone 134591, R&D Systems) was purchased from R&D Systems. AF647 Goat anti-human IgG(H+L) F(ab′)₂ fragment antibody was purchased from Jackson ImmunoResearch (West Grove, PA, USA). Anti-SARS-COV-2 Coronavirus Spike protein (subunit 1) polyclonal antibody was purchased from SinoBiological (Beijing, China). Anti-SARS-CoV-2 Spike RBD rabbit polyclonal antibody was purchased from SinoBiological (Beijing, China). Anti-His mouse monoclonal antibody IgG1 (clone H-3) was purchased from Santa Cruz Biotechnology (Dallas, TX, USA). Alexa Fluor 488 goat anti-rabbit IgG (H+L) and Alexa Fluor 488 goat anti-mouse IgG1 (γ1) were purchased from Fisher Scientific (Waltham, MA).

Cell lines: 293T, A549, HepG2, and NK-92MI cell lines were purchased from the American Type Culture Collection (ATCC). 293T-hACE2 cell line was a gift from Dr. Abraham Pinter (Rutgers-New Jersey Medical School, PHRI). To maintain the stable expression of hACE2, 293T-hACE2 cells were cultured in DMEM (Corning) supplemented with 10% (v/v) fetal bovine serum (FBS), 100 U/mL Penicillin-Streptomycin (Corning), and 1 μg/mL of puromycin at 37° C. under 5% (v/v) CO₂.

Generation of transient 293T-hACE2-RBD cell line: To establish the transient 293T-hACE2-RBD cell line, 293T-hACE2 cells were transfected with 0.5 μg of SARS-COV-2-RBD plasmid (a gift from Dr. Abraham Pinter) in each well in a 24-well plate (Eppendorf) for 48 hours at 37° C. under 5% (v/v) CO₂. Transfected cells were harvested after 48 hours and stained with primary anti-RBD (SinoBiological) followed by a goat anti-rabbit fluorophore-conjugated secondary antibody to determine the expression of RBD by flow cytometry.

Generation of stable A549-Spike cell line: pcDNA3.1-SARS-COV-2 Spike (Addgene plasmid #145302) was used to clone SARS-COV-2 S gene into the SFG backbone with forward primer TCTAGAGATTACAAGGATGACGACGATAAGTAACTCGAGATCGATCCGGAT TAGTCCAAT (SEQ ID NO: 9) and reverse primer GTCGACGCACTGGACACCTTTTAAAATAG (SEQ ID NO:10) using the In-Fusion Cloning kit (Takara Bio). 293T cells were transfected with 3.75 μg SFG-SARS-COV-2 S, 2.5 μg RDF, and 3.75 μg PegPam3 for 48 hours at 37° C. under 5% (v/v) CO₂. The spike retrovirus supernatant was filtered (0.45 μm) and transduced into A549 cells for an additional 48-72 hours at 37° C. under 5% (v/v) CO₂. After 2-3 days, transduced cells were changed to fresh DMEM (Corning) supplemented with 10% (v/v) fetal bovine serum (FBS), 100 U/mL Penicillin-Streptomycin (Corning). The spike protein expression was determined by flow cytometry by staining the transduced cells with anti-RBD antibody (SinoBiological) followed by a goat anti-rabbit fluorophore-conjugated secondary antibody. A549-Spike cells were cultured for a few days prior to sorting using anti-RBD. Sorted cells were cultured in DMEM supplemented with 10% (v/v) FBS, and 100 U/mL Penicillin-Streptomycin.

Production of pseudotyped SARS-COV-2 viral particles: Briefly, 293T cells were transfected using a lentivirus system with a combination of plasmids including plp1, plp2, pCMV-luciferase-ecoGFp (a gift from Cornell University), and pcDNA 3.1-SARS-COV-2 Spike (Addgene plasmid #145032) for 72 hours at 37° C. under 5% (v/v) CO₂. The pseudovirus was then filtered (0.45 μm). To confirm the presence of pseudotyped SARS-COV-2 viral particles, the filtered pseudovirus supernatant was used to transfect 293T-hACE2 for 48 hours at 37° C. under 5% (v/v) CO₂. The GFP expression of the transfected 293T-hACE2 cells was observed using an EVOS FL microscope (Life Technologies). The presence of the SARS-COV-2 pseudovirus was further confirmed by flow cytometry, transfected 293T-hACE2 cells were stained with primary anti-RBD followed by goat anti-rabbit fluorophore-conjugated secondary antibody.

S309-CAR construction and retrovirus production: A codon-optimized DNA fragment was synthesized by GENEWIZ encoding the S309-specific scFv and sub-cloned into the SFG retroviral vector retroviral backbone in-frame with the hinge component of human IgG1, CD28 transmembrane domain, intracellular domain CD28 and 4-1BB, and the ζ chain of the human TCR/CD3 complex. Both the codon-optimized anti-S309 scFv fragment and the SFG vector were digested with restriction endonucleases SalI and BsiWI. SFG-S309 plasmid was transformed into Stb13 chemically competent cells. Maxiprep was performed to enrich DNA concentration for the transfection step.

To produce S309-CAR retrovirus, 293T cells were transfected with 3.75 μg S309-CAR in SFG backbone, 3.75 μg PegPam3, and 2.5 μg RDF. S309-CAR retrovirus was harvested after 48-72 hours, filtered with a 0.45 μm filter, and transduced to NK-92MI cells in a 24-well plate coated with 0.5 μg/ml of RetroNectin diluted in PBS (Clontech). Two days later, cells were transferred to 75 cm² flask (Corning) in complete NK-92MI medium (MEM-α with 12.5% (v/v) FBS, 12.5% (v/v) heat inactivated horse serum, 11 μM BME, 2 μM folic acid, and 20 μM inositol. To determine the expression of CAR or to sort S309-CAR-NK-92MI cell line, cells were stained with anti-CD56 and anti-human IgG (H+L) F(ab′)₂ fragment.

Primary NK cell expansion from peripheral blood: Human blood related work was approved by the Rutgers University Institutional Review Board (IRB). Lymphocyte Separation Medium (Corning) was used to isolate PBMCs from the buffy coats purchased from New York Blood Center. To expand human primary NK cells, 5×10⁶ cells of isolated PBMCs were cocultured with 10×10⁶ cells of 100 Gy-irradiated 221-mIL21 cells in 30 mL RPMI 1640 media (Corning) supplemented with 10% (v/v) FBS, 2 mM L-Glutamine (Corning), 100 U/mL Penicillin-Streptomycin, 200 U/mL IL-2 (PeproTech), and 5 ng/ml IL-15 (Peprotech) in a G-REX 6 Multi-well culture plate (Wilson Wolf) at 37° C. under 5% (v/v) CO₂. Medium was changed every 3-4 days. An automated cell counter (Nexcelom Bioscience, Lawrence, MA, USA) was used to count the total cell numbers. The NK cell purity was determined by staining cells with anti-CD56 and anti-CD3 followed by flow cytometry analysis.

Transduction of expanded NK cells with S309-CAR: The transduction procedure was previously described, briefly, 293T cells were transfected with a combination of SFG-S309, PegPam3, and RDF. S309-CAR retrovirus was harvested after 48-72 hours, filtered, and transduced to Day 4 of expanded primary NK cells in a 24-well plate coated with 0.5 μg/ml of RetroNectin. Transduced cells were harvested and transferred to a G-Rex well in 30 mL RPMI 1640 media supplemented with 10% (v/v) FBS, 2 mM L-Glutamine, 100 U/mL Penicillin-Streptomycin, 200 U/mL IL-2 (PeproTech), and 5 ng/ml IL-15 (Peprotech). Medium was changed every 3-4 days up to 21 days. Cells were stained for CD56, CD3, and anti-human IgG (H+L) F(ab′)₂ fragment for the determination of NK cell purity and CAR expression, followed by flow cytometry analysis.

S309-CAR and RBD binding assay: To evaluate the binding activity of CR309-CAR to RBD domain of SARS-COV-2 S, S309-CAR or NK-92MI (5×10⁵) cells were incubated with 5 μg of His-gp70-RBD recombinant protein (a gift from Dr. Abraham Pinter) in DPBS buffer (0.5 mM MgCl₂ and 0.9 mM CaCl₂ in PBS) in for 30 minutes on ice. Cells were washed twice with PBS, stained with anti-His in FACS buffer (0.2% FBS in PBS) for 30 minutes on ice and then washed twice again with PBS. Cells were then stained with goat anti-mouse (IgG1) secondary antibody in FACS buffer for 30 minutes on ice, washed twice with PBS, and analyzed by Flow Cytometry.

S309-CAR and pseudotyped SARS-COV-2 S viral particles binding assay: S309-CAR, NK-92MI, and 293T-hACE2 (5×10⁵) cells were first equilibrated with BM binding media (complete RPMI-1640 containing 0.2% BSA and 10 mM HEPES pH 7.4). Due to the non-specific binding to the S309-CAR of the secondary antibody, cells were first blocked with anti-human IgG (H+L) F(ab′)₂ fragment for 30 minutes on ice in BM and washed thrice with PBS. Full-length recombinant S protein (Acrobio systems), and S1 subunit recombinant protein (a gift from Dr. Abraham Pinter) were diluted with BM to appropriate concentrations. Filtered pseudotyped SARS-COV-2 S was used immediately following filtration without further dilution. Pseudotyped SARS-COV-2 S, or 1 μg of full-length recombinant S protein, or 1 μg of S1 subunit recombinant protein was added to designated wells of a 96-well V bottom plate. The plate was centrifuged at 600×g for 30 minutes at 32° C., and subsequently incubated at 37° C. at 5% CO₂ for 1 hour. Cells were washed twice with PBS, stained with anti-S1 (SinoBiological) in FACS buffer (2% FBS in PBS) for 30 minutes on ice and washed thrice with PBS. Cells were then stained with goat anti-rabbit secondary antibody in FACS buffer for 30 minutes on ice, washed thrice with PBS, and analyzed by Flow Cytometry.

Flow cytometry analysis: Cells were stained and washed as previously described. Cells were analyzed on a FACS LSRII or an LSR Fortessa flow cytometer. PMT voltages were adjusted and compensation values were calculated before data collection. Data were acquired using FACS Diva software and analyzed using FlowJo software.

CD107a degranulation assay: The CD107a degranulation assay was described previously (Song et al., Nat. Commun. 11:3410, 2020). Briefly, NK-92MI or S309-CAR-NK-92MI or CR3022-CAR-NK-92MI cells (5×10⁴) were cocultured with 1×10⁵ 293T-hACE2, 293T-hACE2-RBD, A549, or A549-Spike cells in the presence of GolgiStop (BD Biosciences) in a V-bottomed 96-well plate in complete RPMI-1640 media at 37° C. under 5% CO₂ for 2 hours. The cells were harvested, washed, and stained for CD3, CD56, and CD107a for 30 minutes, and analyzed by flow cytometry.

Cr⁵¹ release assay: To evaluate the cytotoxic activity of CAR-NK cells, the standard 4-hour Cr⁵¹ release assay was used. Briefly, target cells were labeled with Cr⁵¹ at 37° C. for 2 hours and then resuspended at 1×10^{5/}mL in NK-92MI culture medium with 10% FBS. Then, 1×10⁴ target cells were incubated with serially diluted CAR-NK or NK-92MI cells at 37° C. under 5% CO₂ for 4 hours. After centrifugation, the supernatants were collected and transferred to a 96-well Luma plate and the released Cr⁵¹ was measured with a gamma counter (Wallac, Turku, Finland). The cytotoxicity (as a percentage) was calculated as follows: [(sample−spontaneous release)/(maximum release−spontaneous release)]×100.

Statistical analysis: Data were represented as means±SEM. The statistical significance was determined using a two-tailed unpaired Student t test, a two-tailed paired Student t test, a two-way ANOVA, where indicated. p<0.05 was considered statistically significant.

### Example 2

### Generation and Characterization of S309-CAR-NK-92MI Cells

To develop an NK cell-based immunotherapy for a COVID-19 treatment, the scFv domain of S309 was cloned into an SFG retroviral vector that contains a human IgG1 hinge and CH2—CH3 domain, CD28 transmembrane domain and intracellular domain, 4-1BB-Ligand intracellular domain, and CD3ζ intracellular domain (FIG. 1A). S309-CAR-NK cells were generated in the human NK-92MI cell line. 293T cells were transfected with a combination of plasmids containing S309-CAR in the SFG backbone, RDF, and PegPam3, as previously described (Xiong et al., Mol. Ther. 26:963-975, 2018). The SFG retrovirus particles were then used to transduce NK-92MI cells. After 4-5 days, NK-92MI and S309-CAR cells were stained with CD56 and human IgG (H+L) and the CAR expression was analyzed by flow cytometry. Around 70% of CD56⁺ S309-CAR⁺ NK-92MI cells were observed (FIG. 1B). Then, the subsequent S309-CAR positive NK-92 cells were sorted by flow cytometry to achieve high CAR expression levels (FIG. 1B).

To characterize S309-CAR-NK-92MI cells, the expression of several key immunoreceptors on S309-CAR-NK-92MI cells was examined by flow cytometry. These receptors include TIGIT, LAG-3, TIM-3, KLRGI, CTLA-4, PD-1, CD69, CD8A, NKG2C, CD94, DNAM-1, 2B4, NKG2D, NKp46, and CD16 (FIG. 1C). Overall, the expression of these activating and inhibitory receptors were comparable between parental NK-92MI and S309-CAR-NK-92MI cells, indicating the stable characteristics of NK-92MI at pre-and post-transduction stages.

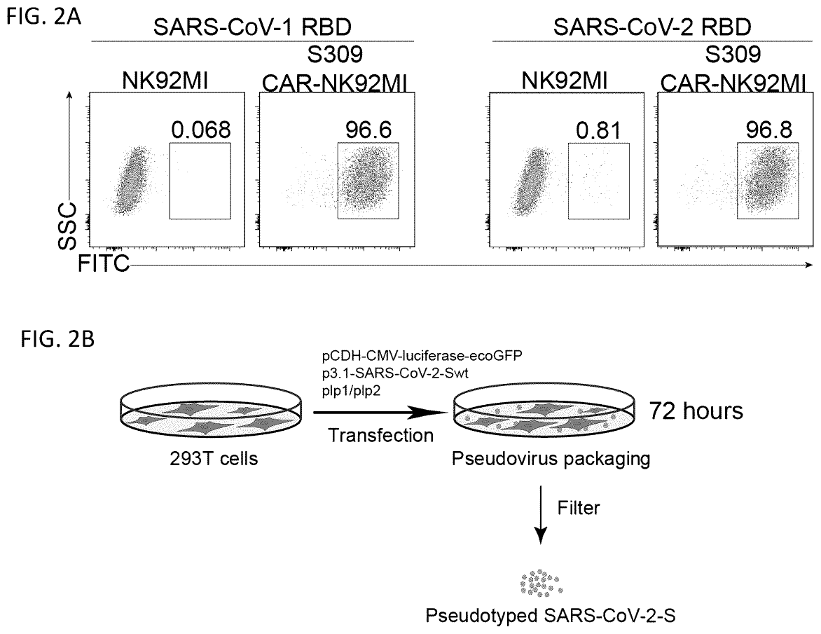

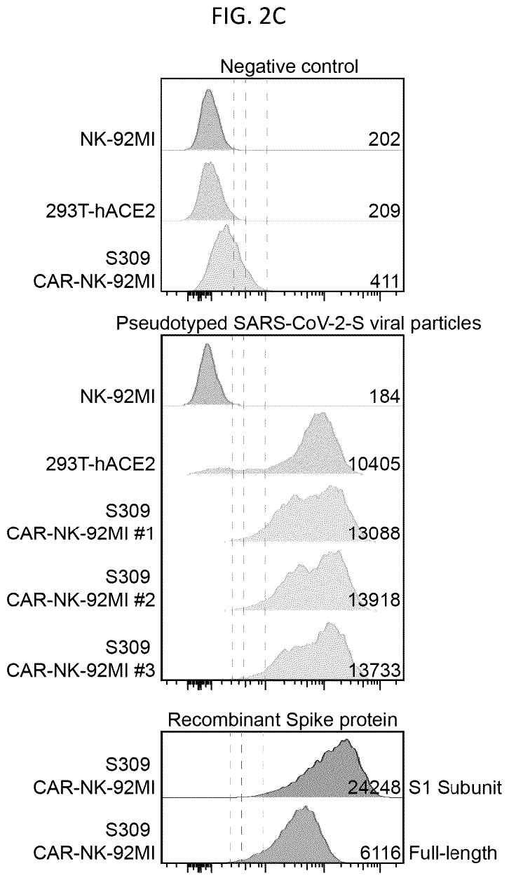

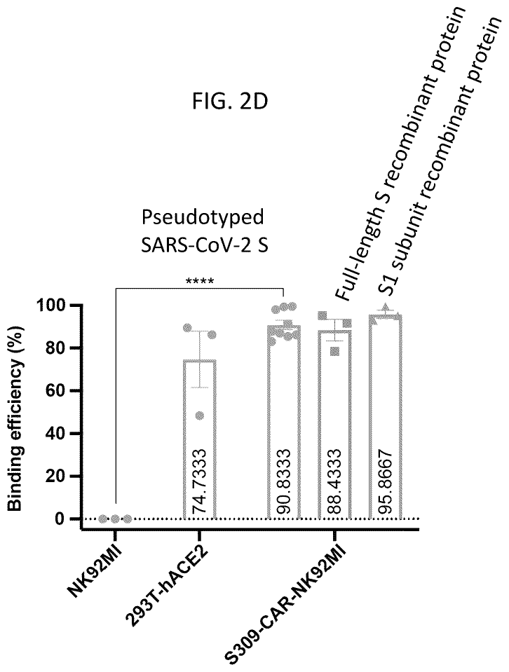

After successful establishment of S309-CAR-NK-92MI cells, the binding ability of S309-CAR-NK cells to the RBD domain of SARS-COV-2 S protein was assessed. Since S309 neutralizing antibody was isolated from memory B cells of a SARS patient, the recombinant His-RBD protein of SARS-COV was included as a positive control. S309-CAR-NK-92MI cells and NK-92MI cells were incubated with the His-RBD of SARS-COV or SARS-COV-2 and the resulting complex was then recognized by anti-His and its corresponding fluorophore-conjugated-secondary antibody. Flow cytometry was employed to evaluate the binding efficiency of S309-CAR to the RBD of S protein from either SARS-COV or SARS-COV-2. S309 recognized and strongly bound to the RBD of both SARS-COV and SARS-COV-2 (FIG. 2A).

However, the partial RBD domain of SARS-COV-2 S may not fully reflect the complexity of SARS-COV-2 viral particles. The binding ability of S309-CAR-NK cells to pseudotyped SARS-COV-2 S viral particles that we generated in the lab was therefore evaluated. Pseudotyped SARS-COV-2 viral particles were produced by transfecting 293T cells with a combination of pCMV-luciferase-ecoGFP, pcDNA3.1-SARS-COV-2 Spike, plp1, and plp2 plasmids. The supernatant containing pseudotyped SARS-COV-2 viral particles were filtered for the pseudovirus binding assay (FIG. 2B). To further confirm the presence of pseudotyped SARS-COV-2 viral particles, the collected supernatant was used to infect 293T-hACE2 cells. The GFP expression of the infected 293T-hACE2 cells was then observed using EVOS florescence microscope in addition to flow cytometry analysis (data not shown).

Previous studies showed that the RBD of Spike protein binds to ACE2 and facilitates SARS-COV-2 entry (Lan et al., Nature 581:215-220, 2020). Thus, 293T-hACE2 was included as a positive control (FIG. 2C). Full-length Spike and RBD-containing S1 subunit recombinant proteins were also included as additional control groups. To evaluate the binding ability of S309-CAR-NK-92MI to the pseudotyped SARS-COV-2 S virus, S309-CAR-NK-92MI, NK-92MI or 293T-hACE2 were incubated with SARS-COV-2 S viral particles, SI subunit, or full-length Spike recombinant protein. The complex can be recognized by anti-S1 subunit antibody and its corresponding fluorophore-conjugated secondary antibody. As expected, S309-CAR-NK-92MI cells were able to bind to the pseudotyped SARS-COV-2 S viral particles with slightly lower binding efficiency than that of recombinant protein groups (FIG. 2C). Surprisingly, S309-CAR-NK cells showed a stronger binding efficiency to the pseudotyped SARS-COV-2 viral particles compared to that of 293T-hACE2 cells, suggesting that S309-CAR-NK-92MI may have superior binding capabilities to the SARS-COV-2 virus compared to the natural receptor, ACE2 (FIG. 2D).

### Example 3

### Specific Killing of Target Cells By S309-CAR-NK Cells

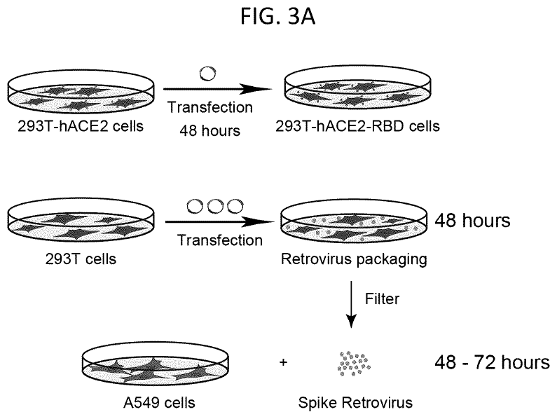

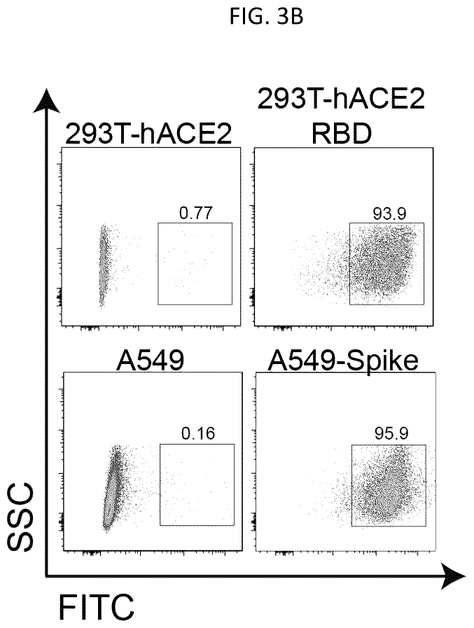

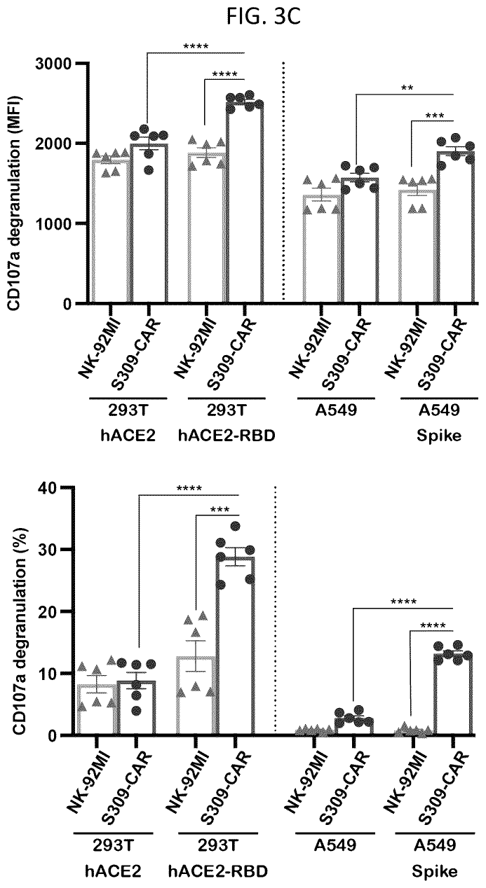

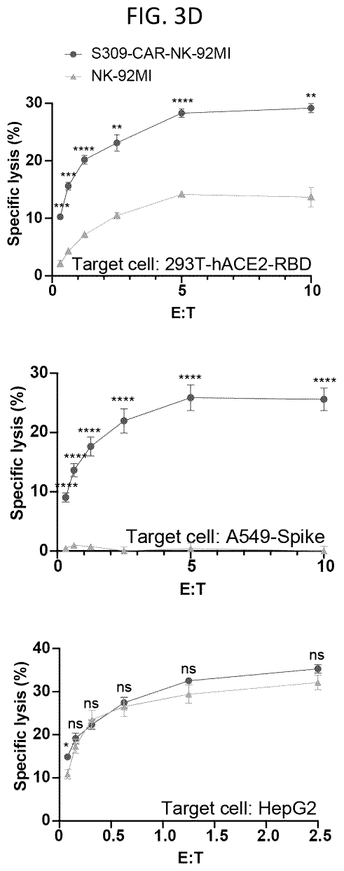

After successful generation of S309-CAR-NK cells and demonstration of recombinant His-RBD protein and pseudotyped SARS-COV-2 S viral particle binding, S309-CAR-NK cells were evaluated for activation by target cells expressing SARS-CoV-2 Spike protein. To test this, two different cell lines expressing the RBD and spike proteins were generated using 293T-hACE2 and A549 cells, respectively. For the generation of transient 293T-hACE2-RBD cells, an RBD encoding plasmid was transfected into 293T-hACE2 cells (a commonly used cell line for studying the SARS-CoV-2 virus) (FIG. 3A). On average, the transfection efficiencies of RBD proteins on 293T-hACE2 cells were greater than 90% as determined by flow cytometry, immunohistochemistry, and immunocytochemistry confocal microscopy (FIG. 3B). For the generation of the stable A549-Spike cell line, the retrovirus packaging system was used to produce Spike retrovirus that was then transduced into A549 cells (a non-small-cell lung carcinoma cell line) (FIG. 3A). The pre-sorting transduction efficiency was around 70% verified by flow cytometry (data not shown). Transduced A549-Spike cells were subsequently sorted to achieve homogeneously high expression levels of Spike proteins (FIG. 3B).

Next, the activation of S309-CAR-NK-92MI cells by 293T-hACE2-RBD or A549-Spike was examined using the CD107a assay. As expected, there was a significant increase in the surface level expression of CD107a molecules on S309-CAR-NK-92MI cells after co-culturing with susceptible 293T-hACE2-RBD or A549-Spike compared to that of wild-type 293T-hACE2 or A549 cells. There was also an increase in total CD107a (percentage and mean fluorescence intensity) on S309-CAR-NK-92 MI cells compared to that of NK-92MI cells (FIG. 3C). Interestingly, there was an increased activation level of S309-CAR-NK-92MI cells when cocultured with 293T-hACE2-RBD compared to A549-Spike.

To evaluate the killing activity of S309-CAR-NK-92MI against SARS-COV-2-protein-expressing target cells, in vitro, the 4-hour Chromium-51 (Cr⁵¹) release assay (a gold standard assay) was used. The data showed that S309-CAR-NK-92MI cells effectively killed both 293T-hACE2-RBD and A549-Spike cells by in vitro Cr51 release assay (FIG. 3D). An irrelevant target cell line, HepG2 (human hepatoma cell line) was used as a negative control, to confirm the specificity of the S309-CAR-NK cells. As expected, no significant difference in the killing activity of S309-CAR-NK-92MI compared to that of wild-type NK-92MI cells (FIG. 3D) was observed. After evaluating the killing function of S309-CAR-NK-92MI cell line, whether the expanded primary S309-CAR-NK cells also have similar killing function against SARS-COV-2-protein-expressing cells was tested. To expand human primary NK cells from peripheral blood (hereinafter PBNK), peripheral blood mononuclear cells (PBMCs) were isolated from buffy coats from healthy donors and cocultured with 100-Gy irradiated 221-mIL21 feeder cells supplemented with 200 U/mL IL-2 and 5 ng/ml IL-15. In parallel, 293T cells were transfected with a combination of plasmids containing S309-CAR in the SFG backbone, RDF, and PegPam3, as previously described (Xiong et al., Mol. Ther. 26:963-975, 2018). The SFG retrovirus particles were used to transduce expanded PBNK cells at Day 4 (FIG. 4A). After 48 hours, primary S309-CAR-NK cells were transferred to a G-Rex plate for continued culturing for 21 days. The NK cell purity and CAR expression were determined using flow cytometry by staining both PBNK and CAR-NK cells with anti-CD56, anti-CD3 and anti-human IgG (H+L). On average, the NK cell purity was around 90% with approximately 80% CAR transduction efficiencies for the S309-CAR-NK (FIG. 4B).

To immunophenotype the expanded primary S309-CAR-NK, both expanded PBNK and S309-CAR-NK were stained cells for various activating and inhibitory receptors, which were determined by flow cytometry. The inhibitory receptors include CTLA4, PD-1, NKG2A, TIGIT, KLRG1, TIM3, and LAG3. The activating receptors include CD16, 2B4, NKG2C NKG2D, DNAM-1, CD94, and NKp46 (FIG. 4C). In general, the expression of these immunomodulatory receptors were comparable between expanded PBNK and S309-CAR-NK cells.

Similar to S309-CAR-NK-92 MI, the 4-hour Cr⁵¹ release assay was used to evaluate the killing function of expanded primary S309-CAR-NK cells. The data showed that S309-CAR-NK cells effectively killed A549-Spike cells compared to expanded PBNK cells (FIG. 4D).

### Example 4

### Comparison of S309-CAR-NK Cells with Prior CAR-NK Cells

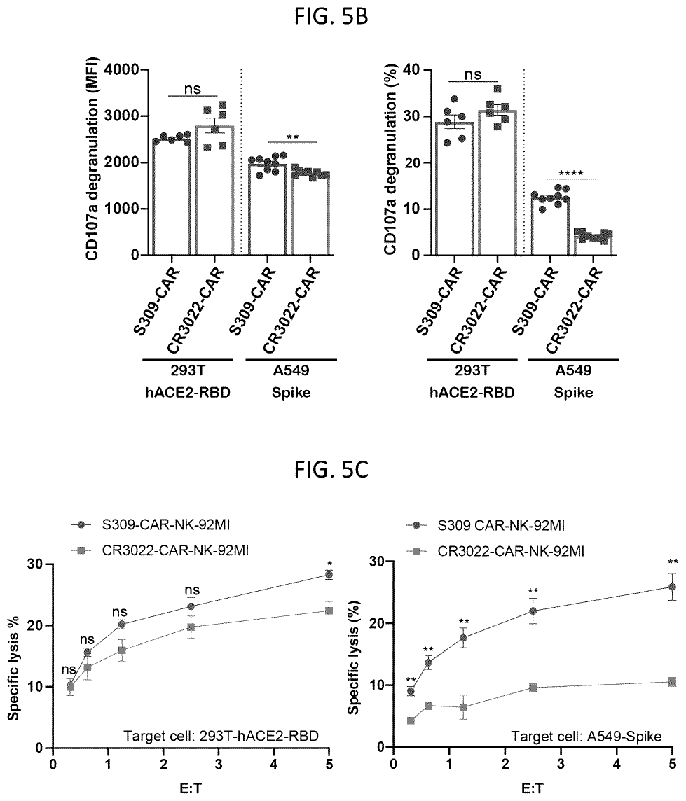

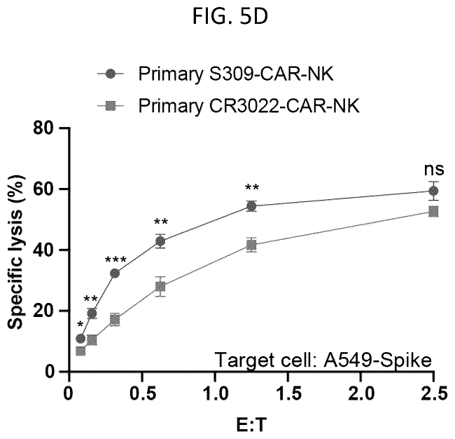

A CR3022-CAR-NK-92MI cell line was previously generated (Ma et al., bioRxiv, doi.org/10.1101/2020.08.11.247320, 2020). Previous studies showed that S309 neutralizing antibody recognizes both open and closed conformations of the SARS-COV-2 S trimer (Pinto et al., Nature 583:290-295, 2020); however, CR3022 can only bind to the open state (FIG. 5A). To confirm these findings, the activation levels of S309-CAR-NK-92MI and CR3022-CAR-NK-92MI cells were compared when cocultured with susceptible 293T-hACE2-RBD or A549-Spike target cells. There was not a significant difference in total CD107a, in both percentages and total mean fluorescence intensity (MFI), against the 293T-hACE2-RBD target cell. However, the expression levels of surface CD107a on CR3022-CAR-NK cells were significantly lower when cocultured with A549-Spike compared to that of S309-CAR-NK cells (FIG. 5B). These data suggest that the conformation of the SARS-COV-2 Spike trimers may play a critical role in CAR recognition and binding ability.

To further evaluate the function of S309-CAR-NK cells, its killing activities were compared to the CR3022-CAR-NK cells. Both 293T-hACE2-RBD and A549-Spike cells were used as susceptible target cells. Consistent with the results in FIG. 5B, a significant decrease in the killing activity of CR3022-CAR-NK cells was observed when A549-Spike cells were the susceptible target cell line in a 4-hour Cr⁵¹ release assay (FIG. 5C). Considering NK-92MI is a cell line and may not fully reflect the function of primary CAR-NK cells, S309-CAR-NK and CR3022-CAR-NK were also generated from expanded PBNK. Primary S309-CAR-NK cells had better killing activities than primary CR3022-CAR-NK (FIG. 5D).

### Example 5

### Recognition of SARS-COV-2 Variants by S309-CAR-NK Cells

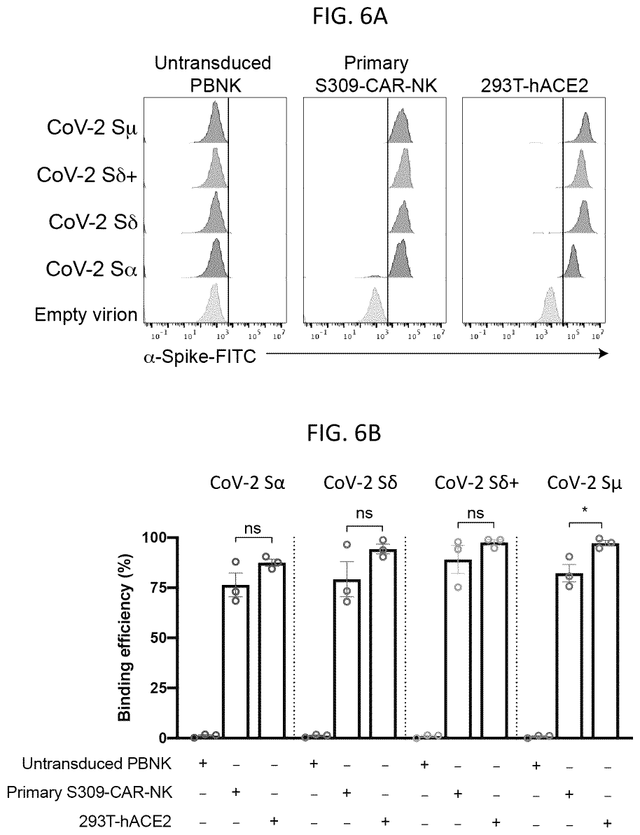

Whether the S309-CAR-NK cells had the ability to recognize different variants of SARS-COV-2 pseudotyped virus was assessed. Different variants of SARS-COV-2 pseudovirus bearing mutations were produced by transfecting 293T cells for 72 hours at 37° C. (Table 2). By incubating S309-CAR-NK cells, un-transduced NK cells as negative control, and 293T-hACE2 cells as positive control with different variants of SARS-COV-2 pseudovirus followed by staining cells with anti-spike and flow cytometry, it was confirmed that S309-CAR-NK cells were effective at binding to the pseudotyped virus of currently existing SARS-COV-2 variants (FIG. 6A).

TABLE 2
Mutations in the spike protein of different SARS-CoV-2
pseudotyped viruses

| Pseudotyped | |
|---|---|
| virus variant | Mutations |
| SARS-CoV-2 Sδ | T19R, T95I, G142D, E156G, ΔF157, ΔR158, |
| | L452R, T478K, D614G, P681R, D950N |
| SARS-CoV-2 Sδ+ | T19R, T95I, A222V, W258L, K417N, L452R, |
| | T478K, E484Q, D614G, P681R, D950N |
| SARS-CoV-2 Sμ | T95I, Y144S, Y145N, R346K, E484K, N501Y, |
| | D614G, P681H, D950N |

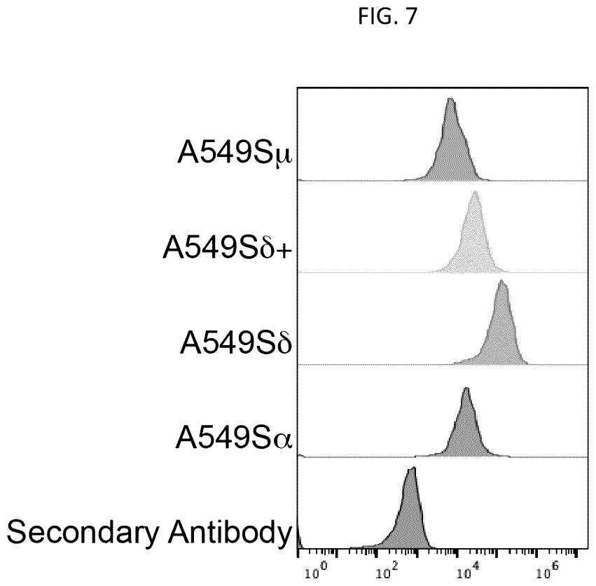

Furthermore, these S309-CAR-NK cells bound to the SARS-COV-2 pseudotyped virus with similar binding efficiency compared to that of 293T-hACE2, suggesting a role for CAR-NK cells in preventing SARS-COV-2 from infecting cells expressing ACE2 receptor (FIG. 6B). The plasmids containing Sα, Sδ, Sδ+, or Sμ were subcloned to SFG expression vector to establish A549 cells expressing the aforementioned mutations (Table 3). Briefly, the PCR product of the target insert was fused into the SFG expression vector using In-Fusion. 293T cells were transfected with plasmids containing Sα, Sδ, Sδ+, or Sμ in SFG expression vector for 48 hours at 37° C. Subsequently, the lentivirus supernatant was collected and transduced into parental A549 cell line for an additional 48 hours followed by flow cytometry to confirm the expression of spike protein (FIG. 7). Target cells expressing the mutated spike protein were subsequently sorted for homogenous expression (data not shown).

TABLE 3
Primer sequences for subcloning

| | | SEQ ID |
|---|---|---|
| Plasmid/Primer | Sequence (5′ - 3′) | NO: |
| pSFG_SARS-CoV2- | TCATGCGGCAGCTGTTGCTCTAGAGATTACA | 11 |
| Sδ_Forward | AGGATGACGACGATAAGTAA | |
| pSFG_SARS-CoV2- | CACCAAGAACACAAACATGTCGACGCACTG | 12 |
| Sδ_Reverse | GACACCTTTTAAAATAGC | |
| pLV_SARS-CoV-2 | GGTGTCCAGTGCGTCGACATGTTTGTGTTCT | 13 |
| Sδ_Forward | TGGTGTTGCTTCCACTG | |
| pLV_SARS-CoV-2 | ATCCTTGTAATCTCTAGAGCAACAGCTGCCG | 14 |
| Sδ_Reverse | CATGAGCAG | |
| pSFG SARS- | TCCTGCGGCAGCTGCTGCTCTAGAGATTACA | 15 |
| CoV2-Sδ+_Forward | AGGATGACGACGATAAGTAA | |
| pSFG_SARS-CoV-2 | GACCAGGAAGACAAACATGTCGACGCACTG | 16 |
| Sδ+_Reverse | GACACCTTTTAAAATAGC | |
| pCAG_SARS- | GGTGTCCAGTGCGTCGACATGTTTGTCTTCC | 17 |
| CoV2-Sδ+_Forward | TGGTCCTGCTGC | |
| pCAG_SARS-CoV- | ATCCTTGTAATCTCTAGAGCAGCAGCTGCCG | 18 |
| 2 Sδ+_Reverse | CAGGAGCAG | |
| pSFG_SARS-CoV-2 | TCCTGCGGCAGCTGCTGCTCTAGAGATTACA | 19 |
| Sμ_Forward | AGGATGACGACGATAAGTAA | |
| pSFG_SARS-CoV-2 | GACCAGGAAGACAAACATGTCGACGCACTG | 20 |
| Sμ_Reverse | GACACCTTTTAAAATAGC | |
| pCAG_SARS-CoV- | GGTGTCCAGTGCGTCGACATGTTTGTCTTCC | 21 |
| 2 Sμ_Forward | TGGTCCTGCTGC | |
| pCAG_SARS-CoV- | ATCCTTGTAATCTCTAGAGCAGCAGCTGCCG | 22 |
| 2 Sμ_Reverse | CAGGAGCAG | |

The cytotoxicity of S309-CAR-NK cells against the sorted target cell lines can be assessed in vitro using degranulation, chromium release, and lipid bilayer assays. The efficacy of S309-CAR-NK cells in preventing SAR-COV2 infection and/or prolonging the survival of NSG-hACE2 mice can also be evaluated.

In view of the many possible embodiments to which the principles of the disclosure may be applied, it should be recognized that the illustrated embodiments are only examples and should not be taken as limiting the scope of the invention. Rather, the scope of the invention is defined by the following claims. We therefore claim as our invention all that comes within the scope and spirit of these claims.

We claim:

1. A chimeric antigen receptor comprising:
(a) an antigen binding domain that specifically binds coronavirus spike protein, comprising an scFv of antibody S309;
(b) a hinge domain;
(c) a transmembrane domain; and
(d) an intracellular domain.

2. The chimeric antigen receptor of claim 1, wherein the antigen binding domain comprises the variable heavy chain (VH) domain complementarity determining region 1 (CDR1), CDR2 and CDR3 amino acid sequences of amino acid positions 47-54, 72-79, and 118-137 of SEQ ID NO: 1, respectively, and the variable light chain (VL) domain CDR1, CDR2 and CDR3 amino acid sequences of amino acid positions 195-201, 219-221, and 258-265 of SEQ ID NO: 1, respectively.

3. The chimeric antigen receptor of claim 1, wherein the antigen binding domain has at least 90% sequence identity to amino acids 22-275 of SEQ ID NO: 1, or comprises or consists of the amino acid sequence of amino acids 22-275 SEQ ID NO: 1.

4. The chimeric antigen receptor of claim 1, wherein the hinge domain comprises an IgG1 domain, the transmembrane domain comprises a CD28 transmembrane domain, and the intracellular domain comprises a CD28 domain, a 4-1BB domain, and a CD3ζ domain.

5. The chimeric antigen receptor of claim 4, wherein the chimeric antigen receptor has at least 90% sequence identity to the amino acid sequence of SEQ ID NO: 1 or comprises or consists of the amino acid sequence of SEQ ID NO: 1.

6. The chimeric antigen receptor of claim 1, further comprising a signal peptide, an interleukin-15 domain, or both.

7. The chimeric antigen receptor of claim 6, wherein the chimeric antigen receptor has at least 90% sequence identity to the amino acid sequence of SEQ ID NO: 4 or comprises or consist of the amino acid sequence of SEQ ID NO: 4.

8. A nucleic acid encoding the chimeric antigen receptor of claim 1.

9. The nucleic acid of claim 8, wherein the antigen binding domain is encoded by a nucleic acid including the variable heavy chain (VH) domain complementarity determining region 1 (CDR1), CDR2 and CDR3 nucleic acid sequences of nucleic acid positions 139-162, 514-237, and 352-411 of SEQ ID NO: 5, respectively, and the variable light chain (VL) domain CDR1, CDR2 and CDR3 nucleic acid sequences of nucleic acid positions 583-603, 655-663, and 772-765 of SEQ ID NO: 5, respectively.

10. The nucleic acid of claim 8, wherein at least a portion of the nucleic acid is codon-optimized.

11. The nucleic acid of claim 8, wherein the nucleic acid has at least 90% sequence identity to the nucleic acid sequence of SEQ ID NO: 5 or SEQ ID NO: 8 or comprises or consists of the nucleic acid sequence of SEQ ID NO: 5 or SEQ ID NO: 8.

12. A vector comprising the nucleic acid of claim 8.

13. The vector of claim 12, wherein the vector is a viral vector.

14. A modified immune cell expressing the chimeric antigen receptor of claim 1.

15. The modified immune cell of claim 14, wherein the chimeric antigen receptor is encoded by a nucleic acid comprising nucleic acid sequence of SEQ ID NO: 5 or SEQ ID NO: 8.

16. The modified immune cell of claim 15, wherein the immune cell is a natural killer (NK) cell or a T cell.

17. A composition comprising the modified immune cell of claim 14 and a pharmaceutically acceptable carrier.
